(19) United States
(12) Patent Application Publication
(10) Pub. No.: US 2025/0242031 A1
(43) Pub. Date: Jul. 31, 2025

(54) POLYMER SALTS FOR IMPROVED DRUG DELIVERY FROM AMORPHOUS SOLID DISPERSIONS

(71) Applicant: Purdue Research Foundation, West Lafayette, IN (US)

(72) Inventors: Lynne Taylor, West Lafayette, IN (US); Qingqing Qi, West Lafayette, IN (US)

(21) Appl. No.: 18/833,637

(22) PCT Filed: Jan. 18, 2023
(86) PCT No.: PCT/US2023/011021

§ 371 Date: Jul. 26, 2024

Related U.S. Application Data
(60) Provisional application No. 63/398,989, filed on Aug. 18, 2022.
(60) Provisional application No. 63/309,780, filed on Feb. 14, 2022.

(51) Int. Cl. A61K 47/34; A61K 9/10; A61K 31/4174; A61K 31/427; A61K 31/4422

(52) U.S. Cl. CPC A61K 47/34; A61K 9/10; A61K 31/4174; A61K 31/427; A61K 31/4422

(57) ABSTRACT

The invention generally relates to polymer salts for improved drug delivery from amorphous solid dispersions. In certain aspects, the invention provides an amorphous solid dispersion (ASD) composition comprising an active pharmaceutical agent (API); and a polymer salt that enables dispersion of the API in an amorphous matrix.

### RELATED APPLICATIONS

The present application claims the benefit of and priority to each of U.S. provisional patent application Ser. No. 63/398,989, filed Aug. 18, 2022, and U.S. provisional patent application Ser. No. 63/309,780, filed Feb. 14, 2022, the content of each of which is incorporated by reference herein in its entirety.

### GOVERNMENT SUPPORT

This invention was made with government support under 1827493 awarded by the National Science Foundation. The government has certain rights in the invention.

### FIELD OF THE INVENTION

The invention generally relates to polymer salts for improved drug delivery from amorphous solid dispersions (ASD).

### BACKGROUND

Enteric polymers have been used historically as tablet coatings to delay drug release until after the formulation has exited the stomach. Recently, these weakly acidic polymers are increasingly being employed in solubility-enhancing amorphous solid dispersion (ASD) formulations, where the drug is molecularly dispersed in a polymer matrix. Enteric film-coating polymers are essentially polymers containing acid groups, namely, polyacids and typically only dissolve in water above pH=5.0-6.0, including methacrylic acid/ethyl acrylate copolymer (preferably in a weight ratio of 1:99 to 99:1), methacrylic acid/methyl methacrylate copolymer (preferably in a weight ratio of 1:99 to 99:1), and methacrylic acid copolymer, hydroxypropyl methylcellulose acetate succinate (HPMCAS), hydroxypropyl methyl phthalate (HPMCP) and cellulose acetate phthalate (CAP), cellulose acetate trimellitate, cellulose acetate succinate, methyl cellulose phthalic acid, hydroxymethyl cellulose ethyl phthalate, hydroxypropyl methyl acetic acid maleic acid ester, hydroxypropyl methyl trimellitate, carboxy methyl ethyl cellulose, polyvinyl butyrate, polyvinyl alcohol acetate phthalate, polyvinyl acetate phthalate (PVAP), poly(acrylic acid) (PAA).

ASD formulation strategy has been increasingly applied for the oral delivery of poor water soluble active pharmaceutical ingredients (APIs), where it is estimated that up to 90% of developmental and approved drugs can be considered to be poorly water soluble. ASD formulations can create supersaturation with a drug-rich phase in nanosized colloidal species and significantly increase the free drug concentration as compared to formulations containing crystalline drug, leading to enhanced oral absorption and improved bioavailability. However, this desired scenario cannot always be attained especially at a higher drug loading, where drug release from an ASD declines dramatically. A low drug loading formulation can lead to increased pill burden, which will negatively impact patient compliance while increasing production and distribution costs. Therefore, strategies to improve drug release at elevated drug loadings are of interest.

It is widely reported that drug release from ASDs of cellulose based acidic polymer including HPMCAS and HPMCP can be very slow even at relatively low drug load, e.g. 10%. And it's also common that drug release declines greatly at elevated drug load, providing incomplete release. Given that low potency drugs are difficult to formulate as ASDs due to the high excipient burden, the present invention provides the solution of using polymer salts to enable higher drug loadings in ASD formulations without compromised release.

Another reported issue of enteric polymers is the observed lag time for disintegration of enteric coated tablets in the small intestine according to several in vivo investigations. In contrast, during in vitro testing in 50 mM pH 6.8 phosphate buffer, coating disintegration is typically rapid. This discrepancy is thought to be due, at least in part, to the low buffer capacity of intestinal fluids, whereby there is a lower pH at the polymer-water surface, reducing the rate of polymer dissolution.

### SUMMARY

The invention recognizes that the salt form of polymers (e.g., enteric polymers), have not been reported of use in the formulation of amorphous solid dispersions (ASD). One strategy that we propose is to use the salt form on an enteric polymer to improve the drug release at higher drug loadings.

In the present invention, we demonstrated that the dissolution rate of polymer salt was the same in 50 and 5 mM phosphate buffer, while the protonated polymer shows a 6-fold reduction in dissolution rate. Furthermore, we demonstrated ASDs based on enteric polymers showed slower and reduced drug release, while ASDs based on the salt form of corresponding enteric polymers retained similar drug release upon changing from 50 to 5 mM phosphate buffer, corresponding to a buffer capacity of 29 and 3 mM·ΔpH¹, respectively.

This is important as salt formation of enteric polymer leads to a polymer and its ASDs that are more robust to buffer capacity variations of the dissolution medium, which shows both intra- and inter-individual variability in vivo and is much lower than for the commonly used in vitro dissolution test medium, 50 mM phosphate buffer.

After extensive investigations, we found that the ionization extent of polymers significantly affects their behavior with respect to the dissolution rate in neat polymer form and further the performance of amorphous solid dispersions where they are used as the major excipient. Surprisingly, we discovered, in this invention, ionized polymers can afford improved drug release from their ASDs, compared with ASDs of nonionized polymers and the same drugs. Furthermore, ionized polymers can provide higher drug loading ASDs with improved release performance than that achievable for the corresponding protonated polymers.

In certain aspects, the invention provides a polymer salt that comprises a main chain and one or more negatively charged carboxylic groups in the main chain, and one or more counterions of said carboxylates. The polymer may be at least one polymer selected from the group consisting of: methacrylic acid/ethyl acrylate copolymer; methacrylic acid/methyl methacrylate copolymer; methacrylic acid copolymer; hydroxypropyl methylcellulose acetate succinate (HPMCAS); hydroxypropyl methyl phthalate (HPMCP); cellulose acetate phthalate (CAP); cellulose acetate trimellitate; cellulose acetate succinate; methyl cellulose phthalic acid; hydroxymethyl cellulose ethyl phthalate; hydroxypropyl methyl acetic acid; maleic acid ester; hydroxypropyl methyl trimellitate; carboxy methyl ethyl cellulose; polyvinyl butyrate; polyvinyl alcohol acetate phthalate; polyvinyl acetate phthalate (PVAP), polyvinyl acetate phthalate (PVAP), poly(acrylic acid) (PAA) and a combination thereof.

The one or more counterions may be at least one salt cation selected from the group consisting of: a Group 1 metal cation; an ammonium comprised of a formula R¹R²R³R⁴N⁺, wherein each of R¹, R², R³ and R⁴ is independently a hydrogen or alkyl group or an aryl group; and a combination thereof. The Group 1 metal cation may be at least one selected from the group consisting of lithium cation (Li⁺), sodium cation (Na⁺), potassium cation (K⁺), rubidium cation (Rb⁺), caesium cation (Cs⁺), and a combination thereof. The ammonium may be selected from NH4+; R⁴ is hydrogen and R¹R²R³N is from meglumine, tris base, triethanolamine, 2-dimethylaminoethanol, triethylamine, ammediol, glucosamine; R¹R²R³R⁴N+ is selected from choline.

In another aspect, the invention provides an amorphous solid dispersion (ASD) composition comprising: an active pharmaceutical agent (API); and a polymer salt of any of the above different formulations. In certain embodiments, the ASD composition further comprising an amino acid or an amino sulfonic acid. In certain embodiments, the polymer salt (e.g., enteric polymer salt) comprises up to about 90 wt % of the ASD composition. In certain embodiments, the API comprises at least about 5 wt % of the ASD composition. In certain embodiments, the API comprises 10 wt % or more of the ASD composition and the polymer salt comprises up to about 90 wt % of the ASD composition.

In certain embodiments, the API is at least one selected from the group consisting of: antihypertensive agents, antianxiety agents, anticlotting agents, anticonvulsant agents, blood glucose-lowering agents, decongestant agents, antihistamine agents, antitussive agents, antineoplastic agents, beta blocker agents, anti-inflammatory agents, antipsychotic agents, cognitive enhancer agents, anti-atherosclerotic agents, cholesterol-reducing agents, anti-obesity agents, autoimmune disorder agents, anti-impotence agents, antibacterial agents, antifungal agents, hypnotic agents, anti-Parkinsonism agents, anti-Alzheimer's disease agents, antibiotic agents, anti-depressant agents, antiviral agents, glycogen phosphorylase inhibitor agents, cholesterol ester transfer protein inhibitor agents, and a combination thereof. In certain preferred embodiments, the API is at least one selected from the group consisting of miconazole, or ritonavir, or clotrimazole, felodipine, and a combination thereof.

In another aspect, the invention provides methods for making a polymer salt (e.g., enteric polymer salt), the method comprising: providing a polymer, adding a base to react with one or more carboxylic acid groups in a main chain of the polymer to thereby convert the polymer into an polymer salt, wherein the base is used in an amount of 0.025 equiv to 1.0 equiv, in relation to one equivalent of carboxylic acid groups in the polymer. Base is at least one selected from inorganic bases, organic bases. In certain embodiments, a salt of a zwitterion is used as the base, which is selected from a salt of an amino acid or an amino sulfonic acid. In certain embodiments, polymer salt is made in a one-pot manner where base is added into a mixture of polymer and zwitterion, which is selected from a salt of an amino acid or an amino sulfonic acid. In certain embodiments, the method is performed without an isolation step.

In another aspect, the invention provides methods for making a polymer salt ASD, the method comprising: making a polymer salt using any of the above described methods; and combining the produced polymer salt with an API to form an ASD composition.

The polymer may be at least one polymer selected from the group consisting of: methacrylic acid/ethyl acrylate copolymer; methacrylic acid/methyl methacrylate copolymer; methacrylic acid copolymer; hydroxypropyl methylcellulose acetate succinate (HPMCAS); hydroxypropyl methyl phthalate (HPMCP); cellulose acetate phthalate (CAP); cellulose acetate trimellitate; cellulose acetate succinate; methyl cellulose phthalic acid; hydroxymethyl cellulose ethyl phthalate; hydroxypropyl methyl acetic acid; maleic acid ester; hydroxypropyl methyl trimellitate; carboxy methyl ethyl cellulose; polyvinyl butyrate; polyvinyl alcohol acetate phthalate; polyvinyl acetate phthalate (PVAP), poly(acrylic acid) (PAA) and a combination thereof.

The one or more counterions may be at least one salt cation selected from the group consisting of: a Group 1 metal cation; ammonium comprises a formula R¹R²R³R⁴N⁺, wherein each of R¹, R², R³ and R⁴ is independently a hydrogen or alkyl group or an aryl group; and a combination thereof. The Group 1 metal cation may be at least one selected from the group consisting of lithium cation (Li⁺), sodium cation (Na⁺), potassium cation (K⁺), rubidium cation (Rb⁺), caesium cation (Cs⁺), and a combination thereof. The ammonium may be selected from NH4+; R⁴ is hydrogen and R¹R²R³N is from meglumine, tris base, triethanolamine, 2-dimethylaminoethanol, triethylamine, ammediol, glucosamine; R¹R²R³R⁴N+ is selected from choline.

The API may be at least one selected from the group consisting of: antihypertensive agents, antianxiety agents, anticlotting agents, anticonvulsant agents, blood glucose-lowering agents, decongestant agents, antihistamine agents, antitussive agents, antineoplastic agents, beta blocker agents, anti-inflammatory agents, antipsychotic agents, cognitive enhancer agents, anti-atherosclerotic agents, cholesterol-reducing agents, anti-obesity agents, autoimmune disorder agents, anti-impotence agents, antibacterial agents, antifungal agents, hypnotic agents, anti-Parkinsonism agents, anti-Alzheimer's disease agents, antibiotic agents, anti-depressant agents, antiviral agents, glycogen phosphorylase inhibitor agents, cholesterol ester transfer protein inhibitor agents, and a combination thereof. In certain preferred embodiments, the API is at least one selected from the group consisting of miconazole, or ritonavir, or clotrimazole, felodipine, and a combination thereof. In certain embodiments, the API comprises 10 wt % or more of the ASD composition.

In another aspect, the invention provides an amorphous solid dispersion (ASD) composition made by the above described methods.

Other aspects of the invention provide:
Applications of enteric polymer salts of any of the embodiments of the invention or their mixtures in ASD formulations.
Applications of enteric polymers of any of the embodiments of the invention or their mixtures in ASD formulations in the presence of inorganic base or their mixture. Such inorganic base can be NaHCO₃, Na₂CO₃, KHCO₃, K₂CO₃, NH₄HCO₃, (NH₄)₂CO₃, NaOH or KOH. The preferred molar ratio of inorganic base to the total amount of acids in the corresponding polymers are 0.05 to 1.0 when bases are selected from NaHCO₃, KHCO₃, NH₄HCO₃, NaOH or KOH. The ratio is preferred to be 0.025 to 0.5 when bases are selected from Na₂CO₃, K₂CO₃, (NH₄)₂CO₃.
Applications of enteric polymers of any of the embodiments of the invention or their mixtures in ASD formulations of APIs in the presence of appropriate amount of amines, amino acid salts or amino sulfuric acid salts.
Applications of amino acid salts or amino sulfuric acid salts or their mixtures in ASD formulations.
A method for making a polymer salt ASD, the method comprising: polymer salt of any of the embodiments of the invention; combing with a zwitterion and an API to form an ASD composition.
Zwitterion of any of the embodiments of the invention is preferred in an amount of 0.02 equiv to 1.0 equiv in relation to one equivalent of carboxylic acid groups of polymer salt in its fully protonated form.
Zwitterion any of the embodiments of the invention involving amino acids, amino sulfuric acids, trimethylglycine, carnitine, acetylcarnitine or their mixtures.
Applications of zwitterion in ASD formulations. The Zwitterion may be selected from amino acids, amino sulfuric acids, trimethylglycine, carnitine, acetylcarnitine or their mixtures.

### BRIEF DESCRIPTION OF THE DRAWINGS

FIG. 1 shows a metathesis reaction for the preparation of polymer salts.

FIG. 2 is ¹H NMR of HP-50-Na in DMSO-d₆.

FIG. 3 is ¹H NMR of HPMCP-50 in DMSO-d₆.

FIG. 4 is ¹H NMR of HP-50-Na in D₂O.

FIG. 5 is Fourier Transform Infrared (FTIR) Spectroscopy of HP-50-Na and HP-50.

FIG. 6 is TGA profile of HP-50-Na.

FIG. 7 is ¹H NMR of PTBA in DMSO-d₆.

FIG. 8 shows PTHAM preparation.

FIG. 9 shows PTEA preparation.

FIG. 10 shows PMP preparation.

FIG. 11 shows PDIP preparation.

FIG. 12 shows PBTM preparation.

FIG. 13 shows PBTP preparation.

FIG. 14 shows HP-55-Na preparation.

FIG. 15 shows PVAP-Na preparation

FIG. 16 shows HP-50-Proline-Na preparation.

FIG. 17 shows HP-50-K preparation.

FIG. 18 shows AS-LF-Na preparation.

FIG. 19 shows CAP-Na preparation.

FIG. 20 shows release profiles of HP-50-Na and HP-50.

FIG. 21 shows that normalized dissolution rate of pre-ionized and protonated polymers in 50 mM pH 6.8 sodium phosphate buffer.

FIG. 22 shows the water sorption of neat polymers, which was measured gravimetrically at various time intervals for up to 96 hours.

FIG. 23 shows a full summary of all tested neat polymers.

FIG. 24 shows normalized polymer release rate of HP-50-Na and HP-50 at different buffer capacities. 50 mM and 5 mM pH 6.8 sodium phosphate buffer were used here.

FIG. 25 shows normalized HP-50 release rate of HP-50-BIS-TRIS at different ionization extent.

FIGS. 26A-B show impact of polymer type on drug release at 20% drug loading.

FIG. 27 shows drug release percentage of 60% drug loading ASD at 30 min is over 60%.

FIGS. 28A-B show release profiles of HP-50, HP-50-Na, HP-50-Meglumine and HP-50-Proline-Na ASD in 5 mM and 50 mM phosphate buffer.

FIG. 29 shows release profile of HP-50-Na-Miconazole ASD in pH 1.6 and pH 6.8 phosphate buffer.

FIG. 30 shows powder dissolution of miconazole-HP-50-Na ASD in pH 1.6 phosphate buffer.

FIG. 31 shows release profiles of HP-50-Ammonium-Miconazole ASDs at 20% drug loading.

FIG. 32 shows dissolution of HP-50-Proline-Na-Miconazole ASD at 20% drug loading. HP-50-TEA-Miconazole, HP-50-Na-Miconazole and HP-50-Miconazole ASDs at 20% drug loading were used here as references.

FIG. 33 shows dissolution of HP-50-Proline-Na-Miconazole ASD at different drug loadings. HP-50-Na (black) and HP-50 (red) ASDs at 20% drug loading used as references.

FIG. 34 shows miconazole release percentage of HP-50-Proline-K-Miconazole ASDs (65% drug loading) at 40 min was over 70%.

FIG. 35 shows dissolution of CAP-Ammonium-Miconazole ASD.

FIG. 36 shows felodipine release profile of PVAP-DMG-Na-Felodipine ASDs at different drug loadings.

FIG. 37 shows ledipasvir release profile of PVAP-DMG-Na-Ledipasvir ASDs at different drug loadings.

### DETAILED DESCRIPTION

The invention generally relates to pre-ionized polymers (e.g., enteric polymers) and their applications in amorphous solid dispersions (ASD) for improved drug release.

In the present invention, polymers were ionized with a calculated partial or stoichiometric amount of bases as indicated in each case. The solvents for the neutralization of polymers can be selected from water or organic solvents, for example, alcohols including methanol, ethanol, isopropanol; chlorinated hydrocarbons including dichloromethane (DCM), acetone; ethers including tetrahydrofuran (THF), 2-Methyltetrahydrofuran (2-MeTHF), or their mixtures as cosolvents. Neutralization temperature ranges from −30 to 100° C.

Carboxylic acid groups in these polymers were neutralized by different basic molecules affording corresponding cations for ionized carboxylic acid groups. Basic molecules include hydroxide, alkoxide, carbonate and bicarbonate of alkali metals, and ammoniums; inorganic amines as ammonia; organic amines include primary, secondary and tertiary amines; amino acid salts, salts of amino sulfonic acid;

Hydroxide, carbonate and bicarbonate of alkali metals are selected but not limited from NaOH, KOH, Na₂CO₃, K₂CO₃, NaHCO₃, KHCO₃, NH₄HCO₃, (NH₄)₂CO₃; Organic amines are selected but not limited from meglumine, tris base, triethanolamine, 2-dimethylaminoethanol, triethylamine, ammediol, glucosamine; amino alcohols; amino acids for amino acid salts are selected from but not limited to proline, sarcosine, dimethylglycine (DMG), bicine, tricine; amino sulfonic acids for salts of amino sulfonic acid are selected from but not limited to MOPSO (2-hydroxy-3-morpholinopropanesulfonic acid), MOPS (3-(N-morpholino)propanesulfonic acid), CHES (N-cyclohexyl-2-aminoethanesulfonic acid), BES (N,N-bis(2-hydroxyethyl)taurine), TES (N-[tris(hydroxymethyl)methyl]-2-aminoethanesulfonic acid); Organic ammoniums are selected from choline, tetramethylammonium, tetrabutylammonium. Amino acids and amino sulfonic acids themselves can also be used to ionize polymers in a one-pot procedure. For example, amino acid or amino sulfonic acid is dissolved first followed by addition of the selected base, after stirring for certain time ranging from minutes to hours, polymers are added. In this case, the amino acid salt or amino sulfonic acid salt is formed in-situ. In addition, the neutralization of polymers can also be realized by mixing polymers, amino acid or amino sulfonic acid first followed by the addition of the appropriate base in one pot.

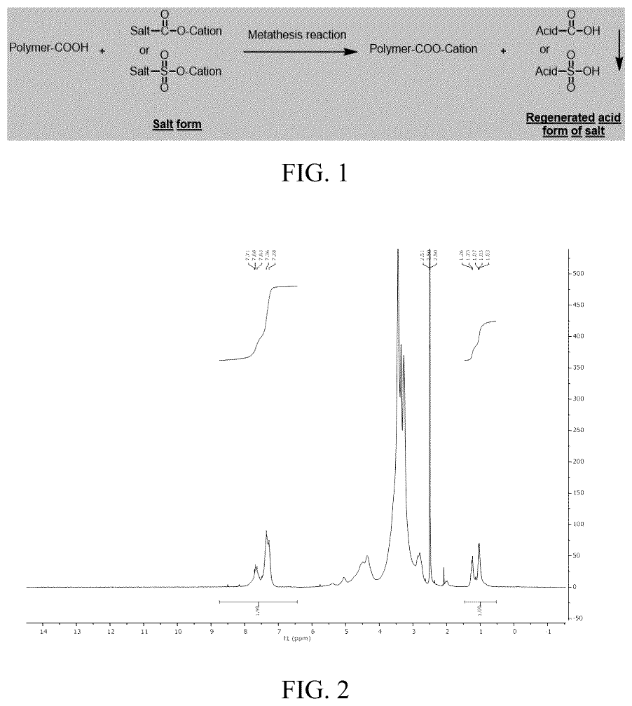

Polymer salts can also be made by metathesis reaction of polymers and salts, during which the proton of the carboxylic acids in the polymer exchange with the cation of the salt to give an ionized polymer and acid form of the salt (FIG. 1). Pure polymer salt solution is separated from the precipitates of newly formed acid either by filtration or centrifugation. Evaporation of the solvents affords the neat polymer salt. Or in other cases, such solution can be used directly for further purpose. Such salts for the metathesis reaction are either commercially available or can be made by reacting their acid form with selected bases. These salts are selected from MOPS sodium, and BES sodium. Acids for the salts are amino acid or amino sulfonic acid, and are selected from taurine, N-methyltaurine, amino acids except for proline and dimethylglycine (DMG).

Water or cosolvents of water can be used for the preparation of polymer salts. Removal of water or cosolvents is achievable by spray drying, evaporation under reduced vacuum or in-lab techniques adapting either rotary evaporation or freeze drying.

Acid content in weight or molar ratio in the polymers can be determined by the reported methods (T. N. Hiew, D. Y. Zemlyanov, L. S. Taylor, Balancing solid-state stability and dissolution performance of lumefantrine amorphous solid dispersions: the role of polymer choice and drug-polymer interactions, Molecular Pharmaceutics, (2022) doi: 10.1021 acs.molpharmaceut.1c00481), the content of each of which is incorporated by reference herein in its entirety. Tested value from the vendor of the polymers can be used as well.

According to one embodiment, the present invention provides pre-ionized polymers, namely, coating polymer salts. Secondly, the present invention provides methods of preparing pre-ionized polymers, namely, e.g., enteric coating polymer salts. Thirdly, the present invention provides methods of ASD preparation of polymers. The method generally includes mixing polymer, amino acid salt or amino sulfonic salt and active pharmaceutical ingredient (API) sequentially. Fourthly, the present invention provides methods of ASD preparation of polymers. The method generally includes mixing a polymer, amino acid or amino sulfonic, selected appropriate base or bases and active pharmaceutical ingredient (API) sequentially. Fifthly, the present invention provides methods of ASD preparation of polymer salt. The method generally includes mixing a polymer salt and active pharmaceutical ingredient (API). Sixthly, the present invention provides dosage forms of ASDs of polymer salts and ASDs prepared as described in third through fifth part.

Polymers listed in the present invention include methacrylic acid/ethyl acrylate copolymer (preferably in a weight ratio of 1:99 to 99:1), methacrylic acid/methyl methacrylate copolymer (preferably in a weight ratio of 1:99 to 99:1), and methacrylic acid copolymer, hydroxypropyl methylcellulose acetate succinate (HPMCAS), hydroxypropyl methyl phthalate (HPMCP) and cellulose acetate phthalate (CAP), cellulose acetate trimellitate, cellulose acetate succinate, methyl cellulose phthalic acid, hydroxymethyl cellulose ethyl phthalate, hydroxypropyl methyl acetic acid maleic acid ester, hydroxypropylmethyl trimellitate, carboxymethylethylcellulose, polyvinyl butyrate, polyvinyl alcohol acetate phthalate, polyvinyl acetate phthalate (PVAP), poly(acrylic acid) (PAA).

Methacrylic acid/ethyl acrylate copolymer (preferably in a weight ratio of 1:99 to 99:1), methacrylic acid/methyl methacrylate copolymer (preferably in a weight ratio of 1:99 to 99:1), and methacrylic acid copolymer, hydroxypropyl methylcellulose acetate succinate (HPMCAS), hydroxypropyl methyl phthalate (HPMCP), cellulose acetate phthalate (CAP) and polyvinyl acetate phthalate (PVAP) are preferable, and hydroxypropyl methylcellulose acetate succinate (HPMCAS), hydroxypropyl methyl phthalate (HPMCP), cellulose acetate phthalate (CAP) and polyvinyl acetate phthalate (PVAP) are particularly preferable.

As a factor showing the solubility of the polymers, the specific examples of the content of each substituent and the composition ratio are preferably the following, but are not limited thereto.
Specific example 1: For HPMCP, methoxy group: 18% by mass to 24% by mass, hydroxypropoxyl group: 5% by mass to 10% by mass, phthalyl group: 27% by mass to 21% by mass;
Specific example 2: For HPMCAS, methoxy group: 20% by mass to 26% by mass, hydroxypropyl group: 5% by mass to 10% by mass, acetate group: 5% by mass to 14% by mass, succinate group: 4% by mass to 18% by mass;
Specific example 3: For CAP, phthalyl group: 30% by mass to 36% by mass, acetyl group: 21.5% by mass to 26% by mass;
Specific example 4: For Eudragit L100, ratio of methacrylic acid and methyl methacrylate units is close to 1 to 1.

The content of the polymers or/and their salts are 10 to 90% by mass in the whole ASD solid where API's ratio is 90 to 10%, preferably 30 to 80% by mass where API's ratio is 70 to 20%, more preferably 50 to 80% by mass where API's ratio is 50 to 20%.

### API

In some embodiments, compositions of the invention may include an API. Formulated as such, the API in various embodiments may be present in an amount of 10 mg to 1000 mg. In some embodiments, the API is present in an amount of 20 mg to 500 mg. In some embodiments, the API is present in an amount of 25 mg to 250 mg. In some embodiments, the API is present in an amount of about 50 mg, about 100 mg or about 125 mg. In some embodiments, a composition of the invention provided herein comprises an API in an amount of from about 1 mg to about 500 mg, from about 10 mg to about 400 mg, from about 10 mg to about 250 mg, from about 5 mg to about 250 mg, from about 25 mg to about 250 mg, from about 25 mg to about 200 mg, from about 50 mg to about 200 mg, from about 50 mg to about 150 mg, from about 75 mg to about 150 mg, from about 100 mg to about 150 mg, from about 125 mg to about 150 mg, from about 75 mg to about 125 mg, from about 100 mg to about 125 mg, from about 75 mg to about 125 mg, from about 50 mg to about 125 mg, from about 25 mg to about 125 mg, from about 75 mg to about 100 mg, from about 50 mg to about 100 mg, from about 25 mg to about 100 mg, from about 5 mg to about 100 mg, from about 50 mg to about 75 mg, from about 25 mg to about 75 mg, from about 5 mg to about 75 mg, from about 50 mg to about 55 mg, from about 5 mg to about 25 mg, from about 10 mg to about 25 mg, from about 5 mg to about 50 mg, from about 10 mg to about 50 mg, or from about 25 mg to about 50 mg, or any ranges therebetween. In some embodiments, a composition of the invention is provided that comprises an API in an amount of at least 10 mg, at least 20 mg, at least 25 mg, at least 30 mg, at least 40 mg, at least 50 mg, at least 60 mg, at least 70 mg, at least 75 mg, at least 80 mg, at least 90 mg, at least 100 mg, at least 110 mg, at least 120 mg, at least 125 mg, at least 130 mg, at least 140 mg, at least 150 mg, at least 160 mg, at least 170 mg, at least 175 mg, at least 180 mg, at least 190 mg, or at least 200 mg. In some embodiments, a composition of the invention comprises an API in an amount of from about 5 mg to about 200 mg. In some embodiments, a composition of the invention is provided that comprises an API in an amount of at most 1000 mg. In some embodiments, the API is present in an amount of at most 750 mg, at most 500 mg, at most 400 mg, at most 300 mg, at most 250 mg, at most 225 mg, at most 200 mg, at most 175 mg, at most 150 mg, at most 125 mg, at most 100 mg, at most 90 mg, at most 80 mg, at most 75 mg, at most 60 mg, at most 55 mg, at most 50 mg, at most 25 mg, or at most 10 mg.

In some embodiments, a composition of the invention includes an API that is present in an amount of from about 1.0 mg to about 1000 mg, including but not limited to about 1.0 mg, 1.5 mg, 2.5 mg, 3.0 mg, 4.0 mg, 5.0 mg, 6.0 mg, 6.5 mg, 7.0 mg, 7.5 mg, 8.0 mg, 8.5 mg, 9.0 mg, 9.5 mg, 10.0, 10.5 mg, 11.0 mg, 12.0 mg, 12.5 mg, 13.0 mg, 13.5 mg, 14.0 mg, 14.5 mg, 15.0 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 36 mg, 36.5 mg, 37 mg, 37.5 mg, 38 mg, 38.5 mg, 39 mg, 39.5 mg, 40 mg, 40.5 mg, 41 mg, 41.5 mg, 42 mg, 42.5 mg, 43 mg, 43.5 mg, 44 mg, 44.5 mg, 45 mg, 45.5 mg, 46 mg, 46.5 mg, 47 mg, 47.5 mg, 48 mg, 48.5 mg, 49 mg, 49.5 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 326 mg, 326.5 mg, 327 mg, 327.5 mg, 328 mg, 328.5 mg, 329 mg, 329.5 mg, 330 mg, 330.5 mg, 331 mg, 331.5 mg, 332 mg, 332.5 mg, 333 mg, 333.5 mg, 334 mg, 334.5 mg, 335 mg, 335.5 mg, 336 mg, 336.5 mg, 337 mg, 337.5 mg, 338 mg, 338.5 mg, 339 mg, 339.5 mg, 340 mg, 340.5 mg, 341 mg, 341.5 mg, 342 mg, 342.5 mg, 343 mg, 343.5 mg, 344 mg, 344.5 mg, 345 mg, 345.5 mg, 346 mg, 346.5 mg, 347 mg, 347.5 mg, 348 mg, 348.5 mg, 349 mg, 349.5 mg, 350 mg, 350.5 mg, 351 mg, 351.5 mg, 352 mg, 352.5 mg, 353 mg, 353.5 mg, 354 mg, 354.5 mg, 355 mg, 355.5 mg, 356 mg, 356.5 mg, 357 mg, 357.5 mg, 358 mg, 358.5 mg, 359 mg, 359.5 mg, 360 mg, 360.5 mg, 361 mg, 361.5 mg, 362 mg, 362.5 mg, 363 mg, 363.5 mg, 364 mg, 364.5 mg, 365 mg, 365.5 mg, 366 mg, 366.5 mg, 367 mg, 367.5 mg, 368 mg, 369.5 mg, 370 mg, 370.5 mg, 371 mg, 371.5 mg, 372 mg, 372.5 mg, 373 mg, 373.5 mg, 374 mg, 374.5 mg, 375 mg, 375.5 mg, 376 mg, 376.5 mg, 377 mg, 377.5 mg, 378 mg, 378.5 mg, 379 mg, 379.5 mg, 380 mg, 380.5 mg, 381 mg, 381.5 mg, 382 mg, 382.5 mg, 383 mg, 383.5 mg, 384 mg, 384.5 mg, 385 mg, 385.5 mg, 386 mg, 386.5 mg, 387 mg, 387.5 mg, 388 mg, 388.5 mg, 389 mg, 389.5 mg, 390 mg, 390.5 mg, 391 mg, 391.5 mg, 392 mg, 392.5 mg, 393 mg, 393.5 mg, 394 mg, 394.5 mg, 395 mg, 395.5 mg, 396 mg, 396.5 mg, 397 mg, 397.5 mg, 398 mg, 398.5 mg, 399 mg, 399.5 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, or 1000 mg.

### Acids

In one aspect, described herein is an amorphous solid dispersion comprising an API and one or more acids. In some embodiments, the amorphous solid dispersion comprises an API, one or more acids, and a hydrophilic high-molecular weight material. In some embodiments, the amorphous solid dispersion comprises an API, a first acid, a second acid, and a hydrophilic high-molecular weight material. In some embodiments, the API is at least partially protonated.

In some embodiments, an amorphous solid dispersion disclosed herein comprises one or more organic acids. In some embodiments, the organic acid has a pKa smaller than 1. In some embodiments, the organic acid has a pKa that is at most 2. In some embodiments, the organic acid has a pKa that is at most 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 4.0, 5.0, 6.0 or 6.5. In some embodiments, the organic acid is completely ionized. In some embodiments, the one or more organic acids excludes acetic acid.

In some embodiments, the amorphous solid dispersion comprises an API, one or more acids, and a hydrophilic high-molecular weight material. In some embodiments, one or more acids comprises a first acid with a pKa of at most 2 and a second acid with a pKa of greater than 2. In some embodiments, the first acid is an organic acid. In some embodiments, the first acid is oxalic acid, maleic acid, trichloroacetic acid, dichloroacetic acid, trifluoroacetic acid, an aliphatic sulfonic acid, or an aromatic sulfonic acid. In some embodiments, the aliphatic sulfonic acid is methanesulfonic acid, methanedisulfonic acid, triflic acid, ethanesulfonic acid, ethanedisulfonic acid, isethionic acid, 2-mercapto-1-ethansulfonic acid, propanesulfonic acid, butanesulfonic acid, benzylsulfonic acid. In some embodiments, the aromatic sulfonic acid is benzenesulfonic acid, tolylsulfonic acid, or naphthalenesulfonic acid. In some embodiments, the first acid is methanesulfonic acid, methanedisulfonic acid, triflic acid, ethanesulfonic acid, ethanedisulfonic acid, isethionic acid, 2-mercapto-1-ethansulfonic acid, propanesulfonic acid, butanesulfonic acid, benzylsulfonic acid, benzenesulfonic acid, tolylsulfonic acid, or naphthalenesulfonic acid.

In some embodiments, the one or more organic acids are present in the amorphous solid dispersion in an amount of from about 0.10% to about 99% by weight of the total composition. In some embodiments, the one or more organic acids are present in the amorphous solid dispersion in an amount of from about 1% to about 80%, from about 1% to about 60%, from about 1% to about 50%, from about 1% to about 25%, from about 1% to about 10%, from about 1% to about 5%, from about 10% to about 80%, from about 10% to about 60%, from about 10% to about 50%, from about 20% to about 80%, from about 20% to about 60%, from about 20% to about 50%, from about 30% to about 80%, from about 30% to about 60%, from about 30% to about 50%, or from about 30% to about 40% by weight of the total composition. In some embodiments, the one or more organic acids are present in the amorphous solid dispersion in an amount of about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, or about 45% by weight of the total composition. In some embodiments, the one or more organic acids are present in the amorphous solid dispersion in an amount of about 1.0 mg to about 1000 mg, including but not limited to about 5.0 mg, 10.0 mg, 15.0 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, or 350 mg. In some embodiments, the one or more organic acids are present in the amorphous solid dispersion in an amount of 1 mg to 500 mg. In some embodiments, the one or more organic acids are present in an amount of from about from about 10 mg to about 400 mg, 20 mg to about 300 mg, from about 25 mg to about 200 mg, from about 50 mg to about 150 mg, from about 75 mg to about 125 mg, from about 75 mg to about 100 mg, from about 100 mg to about 125 mg, from about 1 mg to about 200 mg, or from about 50 mg to about 200 mg. In some embodiments, the one or more organic acids are present in an amount of 25 mg to 250 mg. In some embodiments, the one or more organic acids are present in an amount of 150 mg to 250 mg. In some embodiments, the one or more organic acids are present in an amount of 150 mg to 200 mg. In some embodiments, the one or more organic acids are present in an amount of 50 mg to 200 mg.

In some embodiments, the one or more organic acids are present in a molar ratio to the API of greater than 0.5:1, greater than 1:1, greater than 1.5:1, greater than 2:1, greater than 2.5:1, or greater than 3:1. In some embodiments, the one or more organic acids are present in a molar ratio to the API of about 0.5:1 to about 1:1, about 0.5:1 to about 1.5:1, about 0.5:1 to about 2:1, about 0.5:1 to about 2.5:1, about 0.5:1 to about 3:1, about 1:1 to about 1.5:1, about 1:1 to about 2:1, about 1:1 to about 2.5:1, about 1:1 to about 3:1, about 1.5:1 to about 2:1, about 1.5:1 to about 2.5:1, about 1.5:1 to about 3:1, about 2:1 to about 2.5:1, about 2:1 to about 3:1, or about 2.5:1 to about 3:1.

In some embodiments, an amorphous solid dispersion described herein comprises an API, one or more acids, and a hydrophilic high-molecular weight material. In some embodiments, one or more acids comprises a first acid with a pKa of at most 2 and a second acid with a pKa of greater than 2. In some embodiments, the molar ratio of the first acid to API is present in a molar ratio to the API of about 0.1:1 to about 10:1, about 0.5:1 to about 5:1, about 0.5:1 to about 3:1, about 0.5:1 to about 1:1, about 0.5:1 to about 1.5:1, about 0.5:1 to about 2:1, about 0.5:1 to about 2.5:1, about 0.5:1 to about 3:1, about 1:1 to about 1.5:1, about 1:1 to about 2:1, about 1:1 to about 2.5:1, about 1:1 to about 3:1, about 1.5:1 to about 2:1, about 1.5:1 to about 2.5:1, about 1.5:1 to about 3:1, about 2:1 to about 2.5:1, about 2:1 to about 3:1, or about 2.5:1 to about 3:1. In some embodiments, the molar ratio of the second acid to API is present in a molar ratio to the API of about 0.1:1 to about 10:1, about 1:1 to about 8:1, about 2:1 to about 7:1, about 4:1 to about 7:1, about 0.5:1 to about 3:1, about 0.5:1 to about 1:1, about 0.5:1 to about 1.5:1, about 0.5:1 to about 2:1, about 0.5:1 to about 2.5:1, about 0.5:1 to about 3:1, about 1:1 to about 1.5:1, about 1:1 to about 2:1, about 1:1 to about 2.5:1, about 1:1 to about 3:1, about 1.5:1 to about 2:1, about 1.5:1 to about 2.5:1, about 1.5:1 to about 3:1, about 2:1 to about 2.5:1, about 2:1 to about 3:1, or about 2.5:1 to about 3:1. In some embodiments, the mass ratio of the second acid to API is present in a mass ratio to the API of about 0.1:1 to about 10:1, about 0.2:1 to about 5:1, about 0.5:1 to about 3:1, about 0.2:1 to about 1.2:1, about 0.4:1 to about 1:1, about 0.5:1 to about 1:1, about 0.5:1 to about 1.5:1, about 0.5:1 to about 2:1, about 0.5:1 to about 2.5:1, about 0.5:1 to about 3:1, about 1:1 to about 1.5:1, about 1:1 to about 2:1, about 1:1 to about 2.5:1, about 1:1 to about 3:1, about 1.5:1 to about 2:1, about 1.5:1 to about 2.5:1, about 1.5:1 to about 3:1, about 2:1 to about 2.5:1, about 2:1 to about 3:1, or about 2.5:1 to about 3:1.

In some embodiments, an amorphous solid dispersion disclosed herein comprises a first acid and a second acid. In some embodiments, a molar ratio of the second acid to the first acid is from about 0.05:1 to about 20:1. In some embodiments, the molar ratio of the second acid to the first acid is from about 0.5:1 to about 10:1. In some embodiments, the molar ratio of the second acid to the first acid is from about 1:1 to about 4:1. In some embodiments, the molar ratio of the second acid to the first acid is about 2:1. In some embodiments, a molar ratio of the API to the first acid is about 0.1:1 to about 10:1. In some embodiments, a molar ratio of the API to the first acid is from about 0.2:1 to about 5:1 or from about 0.5:1 to about 2:1. In some embodiments, a molar ratio of the API to the first acid is about 1:1. In some embodiments, a mass ratio of the API to the second acid is about 0.05:1 to about 20:1. In some embodiments, a mass ratio of the API to the second acid is from about 0.1:1 to about 5:1 or from about 0.2:1 to about 1:1. In some embodiments, a mass ratio of the API to the second acid is about 0.5:1.

In some embodiments, the first acid has a pKa smaller than 1. In some embodiments, the first acid has a pKa that is at most 2. In some embodiments, the first acid is an organic acid. In some embodiments, the first acid is an inorganic acid. In some embodiments, the second acid is an organic acid. In some embodiments, the second acid is an inorganic acid. In some embodiments, both the first and the second acids are organic acids. In some embodiments, the first acid is present in the amorphous solid dispersion and/or in the pharmaceutical composition in an amount of 5 mg to 200 mg. In some embodiments, the first acid is present in an amount of from about 10 mg to about 100 mg, from about 15 mg to about 50 mg, from about 20 mg to about 40 mg, from about 20 mg to about 30 mg, or from about 25 mg to about 30 mg. In some embodiments, the second acid is present in the amorphous solid dispersion in an amount of 5 mg to 400 mg. In some embodiments, the second acid is present in an amount of from about 10 mg to about 400 mg, from about 20 mg to about 300 mg, from about 50 mg to about 200 mg, from about 50 mg to about 150 mg, from about 50 mg to about 100 mg, from about 30 mg to about 60 mg, from about 25 mg to about 75 mg, or from about 100 mg to about 200 mg.

### High Molecular Weight Material

In some embodiments, the compositions of the invention may optionally include a hydrophilic high-molecular weight material, which if present, may be present in the disclosed amorphous solid dispersion in an amount of 5% to 90% of a total weigh of the amorphous solid dispersion. In some embodiments, the compositions of the invention may optionally include a hydrophilic high-molecular weight material, which if present, may be present in the disclosed amorphous solid dispersion in an amount of 5% to 80% of a total weigh of the amorphous solid dispersion. In some embodiments, the hydrophilic high-molecular weight material is present in an amount of from about 10% to about 60%, from about 30% to about 50%, from about 5% to about 60%, from about 20% to about 50%, from about 20% to about 40%, from about 25% to about 35%, from about 28% to about 32%, from about 25% to about 30%, or from about 20% to about 30% of a total weigh of the amorphous solid dispersion. In some embodiments, the hydrophilic high-molecular weight material comprises from about 20% to about 50% of the total weight of the amorphous solid dispersion. In some embodiments, the hydrophilic high-molecular weight material comprises from about 20% to about 40% of the total weight of the amorphous solid dispersion. In some embodiments, the hydrophilic high-molecular weight material is present in an amount of about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% of a total weigh of the amorphous solid dispersion.

In some embodiments, the hydrophilic high-molecular weight material is present in the disclosed pharmaceutical composition in an amount of 5% to 80% of a total weigh of the pharmaceutical composition. In some embodiments, the hydrophilic high-molecular weight material is present in an amount of from about 10% to about 60%, from about 30% to about 50%, from about 5% to about 60%, from about 20% to about 50%, from about 20% to about 40%, from about 25% to about 35%, from about 28% to about 32%, from about 25% to about 30%, or from about 20% to about 30% of a total weigh of the pharmaceutical composition. In some embodiments, the hydrophilic high-molecular weight material comprises from about 20% to about 50% of the total weight of the pharmaceutical composition. In some embodiments, the hydrophilic high-molecular weight material comprises from about 20% to about 40% of the total weight of the pharmaceutical composition. In some embodiments, the hydrophilic high-molecular weight material is present in an amount of about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, or about 40% of a total weigh of the pharmaceutical composition.

In some embodiments, a hydrophilic high-molecular weight material is present in the disclosed pharmaceutical composition as an excipient. In some embodiments, a hydrophilic high-molecular weight material is present in the disclosed pharmaceutical composition as an excipient of the amorphous solid dispersion in an amount of about 0.10% to about 90% by weight, including about 0.1% to about 10%, about 3% to about 8%, about 1%, about 2%, about 3%, about 5%, about 6%, about 7%, about 8%, about 9% or about 10% by weight of the pharmaceutical composition. In some embodiments, a hydrophilic high-molecular weight material is present in the disclosed pharmaceutical composition as an excipient of the amorphous solid dispersion in an amount of about 5% by weight of the pharmaceutical composition.

### Other Excipients and Additives

The present disclosure may also relate to pharmaceutical compositions and methods of administering thereof. In such embodiments, the invention then provides a pharmaceutical compositions comprising an amorphous solid dispersion comprising an API. In some embodiments, the pharmaceutical compositions comprise one or more excipients or additives. In some embodiments, the pharmaceutical compositions comprise an API, one or more acids, and a first acid, wherein the first acid is organic; a second acid; and a high-molecular weight material. In some embodiments, the API is at least partially protonated. In some embodiments, the API is in a salt or solvate form. In some embodiments, the one or more acids comprise two organic acids.

Excipients and additives that can be used in a described pharmaceutical composition include additives well known in the art. Such additives include, but are not limited to, anti-adherents (anti-sticking agents, glidants, flow promoters, lubricants) (e.g., talc, magnesium stearate, fumed silica (Carbosil, Aerosil), micronized silica (Syloid No. FP 244, Grace U.S.A.), polyethylene glycols, surfactants, waxes, stearic acid, stearic acid salts, stearic acid derivatives, starch, hydrogenated vegetable oils, sodium benzoate, sodium acetate, leucine, PEG-4000 and magnesium lauryl sulfate) anticoagulants (e.g., acetylated monoglycerides), antifoaming agents (e.g., long-chain alcohols and silicone derivatives), antioxidants (e.g., BHT, BHA, gallic acid, propyl gallate, ascorbic acid, ascorbyl palmitate, 4hydroxymethyl-2,6-di-tert-butyl phenol, tocopherol, etc.), binders (adhesives), i.e., agents that impart cohesive properties to powdered materials through particle-particle bonding, (e.g., matrix binders (dry starch, dry sugars), film binders (PVP, starch paste, celluloses, bentonite, sucrose)), chemical binders (e.g., polymeric cellulose derivatives, such as carboxy methyl cellulose, crospovidone (i.e., cross linked polyvinyl N-pyrrolidone), HPC, hydroxypropyl methylcellulose (HPMC), etc., sugar syrups, corn syrup, water soluble polysaccharides (e.g., acacia, tragacanth, guar, alginates, etc), gelatin, gelatin hydrolysate, agar, sucrose, dextrose, non-cellulosic binders (e.g., PVP, PEG, vinyl pyrrolidone copolymers, pregelatinized starch, sorbitol, glucose, etc.), chelating agents (e.g., EDTA and EDTA salts), coagulants (e.g., alginates) colorants or opaquants, (e.g., titanium dioxide, food dyes, lakes, natural vegetable colorants, iron oxides, silicates, sulfates, magnesium hydroxide and aluminum hydroxide), coolants, (e.g. halogenated hydrocarbons (e.g., trichloroethane, trichloroethylene, dichloromethane, fluorotrichloromethane), diethylether and liquid nitrogen) cryoprotectants (e.g., trehelose, phosphates, gelatin, dextran, mannitol, etc.), diluents or fillers (e.g., lactose, mannitol, talc, magnesium stearate, sodium chloride, potassium chloride, citric acid, spray-dried lactose, hydrolyzed starches, directly compressible starch, microcrystalline cellulose (MCC), cellulosics, sorbitol, sucrose, sucrose-based materials, calcium sulfate, dibasic calcium phosphate and dextrose disintegrants or super disintegrants (e.g., croscarmellose sodium, starch, starch derivatives, clays, gums, cellulose, cellulose derivatives, alginates, crosslinked polyvinylpyrrolidone, sodium starch glycolate and microcrystalline cellulose), hydrogen bonding agents (e.g., magnesium oxide), flavorants or desensitizers, (e.g., spray-dried flavors, essential oils and ethyl vanillin), ion-exchange resins (e.g., styrene/divinyl benzene copolymers, and quaternary ammonium compounds), plasticizers (e.g., polyethylene glycol, citrate esters (e.g., triethyl citrate, acetyl triethyl citrate, acetyltributyl citrate), acetylated monoglycerides, glycerin, triacetin, propylene glycol, phthalate esters (e.g., diethyl phthalate, dibutyl phthalate), castor oil, sorbitol and dibutyl seccate), preservatives, parabens, phenols, benzyl alcohol, and quaternary ammonium compounds), solvents (e.g., alcohols, ketones, esters, chlorinated hydrocarbons and water) sweeteners, including natural sweeteners (e.g., maltose, sucrose, glucose, sorbitol, glycerin and dextrins), and artificial sweeteners (e.g., aspartame, saccharine and saccharine salts) and thickeners (viscosity modifiers, thickening agents) (e.g., sugars, polyvinylpyrrolidone, cellulosics, polymers and alginates).

Additives can also comprise materials such as proteins (e.g., collagen, gelatin, Zein, gluten, mussel protein, lipoprotein), carbohydrates (e.g., alginates, carrageenan, cellulose derivatives, pectin, starch, chitosan), gums (e.g., xanthan gum, gum arabic), spermaceti, natural or synthetic waxes, camuaba wax, fatty acids (e.g., stearic acid, hydroxystearic acid), fatty alcohols, sugars, shellacs, such as those based on sugars (e.g., lactose, sucrose, dextrose) or starches, polysaccharide-based polymers (e.g., maltodextrin and maltodextrin derivatives, dextrates, cyclodextrin and cyclodextrin derivatives), cellulosic-based polymers (e.g., microcrystalline cellulose, sodium carboxymethyl cellulose, hydroxypropyl methylcellulose, ethyl cellulose, hydroxypropyl cellulose, cellulose acetate, cellulose nitrate, cellulose acetate butyrate, cellulose acetate, trimellitate, carboxymethylethyl cellulose, hydroxypropyl methyl cellulose phthalate), inorganics, (e.g., dicalcium phosphate, hydroxyapitite, tricalcium phosphate, talc and titania), polyols (e.g., mannitol, xylitol and sorbitol polyethylene glycol esters) and polymers (e.g., alginates, poly(lactide coglycolide), gelatin, crosslinked gelatin and agar-agar). (156) In some embodiments, the pharmaceutical compositions comprise one or more preservatives. Preservatives can include anti-microbials, antioxidants, and agents that enhance sterility. Exemplary preservatives include ascorbic acid, ascorbyl palmitate, butylatedhydroxyanisole (BHA), Butylatedhydroxytoulene (BHT), propyl gallate, citric acid, EDTA and its salts, erythorbic acid, fumaric acid, malic acid, propyl gallate, sodium ascorbate, sodium bisulfate, sodium metabisulfite, sodium sulfite, parabens (such as methylparaben, ethylparaben, propylparaben, butylparaben and their salts), benzoic acid, sodium benzoate, potassium sorbate, vanillin, and the like. In some embodiments, an amorphous solid dispersion composition or a pharmaceutical composition described herein comprises an antioxidant. In some embodiments, the antioxidant comprises a-tocopherol acetate, acetone sodium bisulfite, acetylcysteine, ascorbic acid, vitamin E, ascorbyl palmitate, BHA, BHT, cysteine, cysteine hydrochloride, d-a-tocopherol (natural or synthetic), dithiothreitol, monothioglycerol, nordihydroguaiaretic acid, propyl gallate, sodium bisulfite, sodium formaldehyde sulfoxylate, sodium metabisulfite, sodium sulfite, sodium thiosulfate, thiourea, or tocopherols. In some embodiments, an antioxidant or mixture of antioxidants are included as part of a solid dispersion. Exemplary antioxidants include but are not limited to BHT, BHA, gallic acid, propyl gallate, ascorbic acid, ascorbyl palmitate, 4hydroxymethyl-2,6-di-tert-butyl phenol, and tocopherol. In some embodiments, a pharmaceutical composition is provided that comprises from about 0.001% to about 10% by weight of the preservative (e.g., antioxidant). In some embodiments, the percent weight of the preservative or antioxidant is from about 0.001% to about 0.01%, about 0.001% to about 0.1%, about 0.001% to about 1%, about 0.001% to about 5%, about 0.001% to about 10%, about 0.01% to about 1%, about 0.01% to about 5%, about 0.01% to about 10%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 10%, about 1% to about 2%, about 1% to about 5%, about 1% to about 6%, about 1% to about 7%, about 1% to about 8%, or about 1% to about 10%.

In some embodiments, the excipients or additives comprise a filler, a binder, a disintegrating agent, a lubricant, an adsorbent, an acid, or a combination thereof. In some embodiments, the filler and/or binder comprises microcrystalline cellulose, crospovidone, lactose, or a combination thereof. In some embodiments, the disintegrating agent comprises microcrystalline cellulose. In some embodiments, the lubricant comprises magnesium stearate (abbreviated MgSt). In some embodiments, the acid comprises an organic acid such as tartaric acid. In some embodiments, the adsorbent is silica. In some embodiments, the pharmaceutical composition comprises microcrystalline cellulose, lactose, crospovidone, magnesium stearate, silicon dioxide, an organic acid, or a combination thereof.

In some embodiments, the weight ratio of the excipients to the API is from about 0.1:1 to about 10:1. In some embodiments, the weight ratio of the excipients to the API is from about 0.5:1 to about 5:1, from about 0.5:1 to about 4:1, from about 0.5:1 to about 3:1, from about 0.6:1 to about 4:1, from about 0.7:1 to about 3:1, from about 0.8:1 to about 2:1, from about 0.9:1 to about 1.1:1, from about 1:1 to about 3:1, from about 1:1 to about 4:1, from about 1:1 to about 5:1, or from about 1:1 to about 6:1.

It should be appreciated that there is considerable overlap between the above listed components in common usage, since a given component is often classified differently by different practitioners in the field, or is commonly used for any of several different functions, or may have differing functions depending on the levels in the composition. Thus, the above-listed components should be taken as merely exemplary, and not limiting, of the types of components that can be included in compositions of the present invention.

In some embodiments, amorphous solid dispersions described herein comprise an API, a surfactant, a non-ionic hydrophilic polymer, and optionally an adsorbent. In some embodiments, the surfactant is selected from polymeric non-ionic surfactants and phospholipids. In some embodiments, the polymeric non-ionic surfactant comprises a block copolymer of polyethylene glycol and polypropylene glycol. In some embodiments, the polymeric non-ionic surfactant is Poloxamer 188. In some embodiments, the surfactant comprises one or more phospholipids. In some embodiments, the surfactant comprises one or more of phosphatidylcholine, phosphatidylethanolamine, phosphatidylinositol, phosphatidylserine, plasmalogen, sphingomyelin, and phosphatidic acid. In some embodiments, the surfactant comprises lecithin.

### Non-Ionic Hydrophilic Polymer

In some embodiments, amorphous solid dispersions described herein comprise a non-ionic hydrophilic polymer. In some embodiments, the non-ionic hydrophilic polymer comprises oligosaccharide, polysaccharide, vinylpyrrolidone-vinyl acetate copolymer (copovidone), polyvinylpyrrolidone (PVP or povidone), polyvinyl alcohol (PVA), polysaccharide, hydroxypropyl methylcellulose (HPMC or hypromellose), hydroxyethyl cellulose (HEC), hydroxypropyl cellulose (HPC), polyethylene oxide, hydroxypropyl-β-cyclodextrin (HP-β-CD), sulfobutylether-O-cyclodextrin, hydroxypropylmethylcellulose acetate succinate (HPMCAS), polyethylene glycol (PEG), polyvinyl caprolactam-polyvinyl acetate-polyethylene glycol graft copolymer (PVAc-PVCap-PEG), or a combination thereof. In some embodiments, the non-ionic hydrophilic polymer comprises HPMC, copovidone, PVP, HP-β-CD, PVA, HPMCAS, PVAc-PVCap-PEG, or a combination thereof. In some embodiments, the non-ionic hydrophilic polymer comprises about 5% to about 70% of the total weight of the amorphous solid dispersion. In some embodiments, the non-ionic hydrophilic polymer comprises from about 5% to about 60%, from about 5% to about 50%, from about 10% to about 50%, from about 10% to about 40%, from about 20% to about 40%, or from about 20% to about 30% of the total weight of the amorphous solid dispersion.

### Adsorbent

Amorphous solid dispersions described herein can comprise an adsorbent. In some embodiments, a disclosed amorphous solid dispersion comprises an API, one or more acids, an adsorbent and a hydrophilic high-molecular weight material. In some embodiments, the excipients or additives of described pharmaceutical compositions comprise an adsorbent. Adsorbents can be solid, porous or super porous adsorption materials. They can comprise numerous micro- or nano-pores within their structures, resulting in very large surface areas, for example, greater than 500 m.sup.2/g. Exemplary adsorbents include, without limitation, silicon dioxide, active carbon, magnesium aluminum silicate, diatomite, microcrystalline cellulose (MCC), silicified microcrystalline cellulose (SMCC), talc, crosslinked povidone, sodium carboxymethylcellulose, sodium carboxymethyl starch, and also sugars or sugar alcohols such as sorbitol, mannitol, lactose, cyclodextrin, and maltodextrin. In some embodiments, the adsorbent is silicon dioxide.

In some embodiments, an adsorbent such as silicon dioxide is present in a pharmaceutical composition described herein in an amount of at least 5 mg, at least 10 mg, 20 mg, 25 mg, 30 mg, 40 mg, 50 mg, 60 mg, 70 mg, 75 mg, 80 mg, 90 mg, 100 mg, 110 mg, 120 mg, 125 mg, 130 mg, 140 mg, 150 mg, 160 mg, 170 mg, 175 mg, 180 mg, 190 mg, or 200 mg. In some embodiments, the adsorbent is present in an amount of about 10 mg, 20 mg, 25 mg, 30 mg, 40 mg, 50 mg, 60 mg, 70 mg, 75 mg, 80 mg, 90 mg, 100 mg, 110 mg, 120 mg, 125 mg, 130 mg, 140 mg, 150 mg, 160 mg, 170 mg, 175 mg, 180 mg, 190 mg, 200 mg, 225 mg, or 250 mg. In some embodiments, the adsorbent is present in an amount of no more than 300 mg, 250 mg, 225 mg, 200 mg, 175 mg, 150 mg, 125 mg, 100 mg, 90 mg, 80 mg, 75 mg, 60 mg, 55 mg, 50 mg, or 25 mg. In some embodiments, the adsorbent is present in an amount of from about 0.1 mg to about 500 mg. In some embodiments, the adsorbent is present in an amount of from about 0.1 mg, about 0.2 mg, about 0.5 mg, about 1 mg, or 2 about 1 mg to about 5 mg, about 10 mg, about 25 mg, about 50 mg, about 100 mg, or about 200 mg. In some embodiments, the adsorbent is present in an amount of from about 1 mg to about 50 mg, from about 1 mg to about 20 mg, from about 1 mg to about 10 mg, or from about 1 mg to about 5 mg.

In some embodiments, the amorphous solid dispersion is granulated and incorporated into a pharmaceutical composition with extra granular additives. In some embodiments, the silicon dioxide is present outside of the amorphous solid dispersion as an extra-granular additive. In some embodiments, silicon dioxide is present in the amorphous solid dispersion as well as being an extra-granular additive.

In some embodiments, an adsorbent such as silicon dioxide is present in the disclosed amorphous solid dispersion. In some embodiments, the adsorbent comprises from about 1% to about 50% of the total weight of the amorphous solid dispersion. In some embodiments, the adsorbent comprises from about 1% to about 40%, from about 1% to about 30%, from about 1% to about 25%, from about 1% to about 20%, from about 1% to about 15%, from about 1% to about 10%, from about 5% to about 40%, from about 5% to about 25%, from about 5% to about 20%, from about 5% to about 15%, or from about 5% to about 10% of the total weight of the amorphous solid dispersion.

In some embodiments, an adsorbent described herein has a median diameter of 1-1000 nm. In some embodiments, an adsorbent described herein has a median diameter of from about 1 nm to about 750 nm, from about 1 nm to about 500 nm, from about 1 nm to about 250 nm, from about 1 nm to about 150 nm, from about 1 nm to about 100 nm, from about 1 nm to about 50 nm, from about 1 nm to about 25 nm, from about 10 nm to about 500 nm, from about 10 nm to about 250 nm, from about 10 nm to about 150 nm, from about 10 nm to about 100 nm, from about 10 nm to about 50 nm, from about 10 nm to about 25 nm, from about 50 nm to about 500 nm, from about 50 nm to about 250 nm, from about 50 nm to about 150 nm, from about 50 nm to about 100 nm, or from about 25 nm to about 50 nm. In some embodiments, an adsorbent described herein has a median diameter of from about 100 nm to about 1000 nm, from about 100 nm to about 750 nm, from about 200 nm to about 1000 nm, from about 200 nm to about 750 nm, from about 500 nm to about 1000 nm, or from about 500 nm to about 750 nm. In some embodiments, an adsorbent described herein has a median diameter larger than 1000 nm.

### Surfactant

In some embodiments, pharmaceutical compositions described herein comprise an API, a hydrophilic polymer, and a surfactant. In some embodiments, the API, hydrophilic polymer, and the surfactant are formulated as an amorphous solid dispersion. In some embodiments, the surfactant is selected from polymeric non-ionic surfactants and phospholipids. In some embodiments, the surfactants are compounds or mixture of compounds comprising a hydrophobic group (usually a hydrocarbon chain) and a hydrophilic group. They may perform one or more roles including solubility enhancer, bioavailability enhancer, stability enhancer, antioxidant and emulsifying agent. Examples of surfactants include, but are not limited to, phospholipids, sucrose esters of fatty acids, polyoxyl stearate, polyoxyethylene hydrogenated castor oil, polyoxyethylene polyoxypropylene glycol, sorbitan sesquioleate, sorbitan trioleate, sorbitan monostearate, sorbitan monopalmitate, sorbitan monolaurate, polysorbate, glyceryl monostearate, sodium lauryl sulfate, sodium dodecyl sulfate, lauromacrogol Arlasolve, Poloxamers, Labrafil, Labrasol, Tween 80, Tocopheryl polyethylene glycol 1000 succinate (simply TPGS or Vitamin E TPGS) and the like.

In some embodiments, the surfactant used in the present disclosure can be a non-ionic surfactant. A non-ionic surfactant has no charged groups in its head. Exemplary nonionic surfactants include, without limitation, fatty alcohols, cetyl alcohol, stearyl alcohol, cetostearyl alcohol, and oleyl alcohol. Exemplary nonionic surfactants include, but are not limited to, polyethylene glycol alkyl ethers (such as octaethylene glycol monododecyl ether, pentaethylene glycol monododecyl ether), polypropylene glycol alkyl ethers, glucoside alkyl ethers (such as decyl glucoside, lauryl glucoside, octyl glucoside), polyethylene glycol octylphenyl ethers (such as Triton X-100), polyethylene glycol alkylphenyl ethers (such as nonoxynol-9), glycerol alkyl esters (such as glyceryl laurate), polyoxyethylene glycol sorbitan alkyl esters (such as polysorbate), sorbitan alkyl esters (such as Spans), cocamide MEA, cocamide DEA, dodecyldimethylamine oxide, block copolymers of polyethylene glycol and polypropylene glycol (such as poloxamers), polyethoxylated tallow amine (POEA), and Tocopheryl polyethylene glycol 1000 succinate (simply TPGS or Vitamin E TPGS). In some embodiments, the surfactant comprises two more repeating units, such as polyoxyalkylene units. In some embodiments, the surfactant is a non-ionic surfactant that comprises polyethylene glycol. In some embodiments, the surfactant is a block copolymer of polyethylene glycol and polypropylene glycol. In some embodiments, the surfactant is a poloxamer such as poloxamer 188.

In some embodiments, the non-ionic surfactant has a number average molecular weight of from about 1000 to about 100,000 Da, 2000 to about 20,000 Da, from about 4000 to about 15,000 Da, from about 6000 to about 12,000 Da, or from about 7000 to about 10,000 Da. In some embodiments, the non-ionic surfactant has a number average molecular weight of from about 7000 to about 10,000 Da. In some embodiments, the non-ionic surfactant has an ethylene glycol content of from about 30 wt % to about 99 wt %, from about 50 wt % to about 95 wt %, from about 60 wt % to about 95 wt %, from about 75 wt % to about 90 wt %, or from about 80 wt % to about 85 wt %. In some embodiments, the non-ionic surfactant has an ethylene glycol content of from about 80 wt % to about 85 wt %.

In some embodiments, the surfactants are selected from fatty acids, phospholipids, sphingolipids, saccharolipids, polyketides, sterol lipids, prenol lipids and the like. In some embodiments, phospholipids are made up of glycerol to which is attached a phosphate group and two fatty acids. Other terms in the art for phospholipids include glycerophospholipids, phosphoglycerides, diacylglycerides and the like.

In some embodiments, phospholipids are selected from glycerophospholipid, sphingolipid, and/or phospholipid derivatives. In some embodiments, glycerophospholipids include, but are not limited to phosphatidylcholine, phosphatidyl ethanolamine, phosphatidyl ethanolamine, phosphatidyl serine, phosphatidyl glycerol, diphosphatidylglycerol, phosphatidylinositol, and mixtures thereof. Phospholipid derivatives according to the present invention include, but are not limited to dioleoylphosphatidylcholine, dimyristoylphosphatidylcholine, dipentadeanoylphosphatidylcholine, dilauroylphosphatidylchoine, dipalmitoylphosphatidylcholine (DPPC), distearoylphosphatidylcholine (DSPC), diarachidonyiphosphatidylcholine (DAPC), dioleoylphosphatidylethanolamine, dipalmitoylphosphatidylethanolamine (DPPE), and distearoylphosphatidylethanolamine (DSPE), disteraoylphosphatidylglycerol (DSPG), phosphatidylinositol, dipalmitoylphosphatidic acid (DPPA), distearoylphosphatidic acid (DSPA), and the like, and mixtures thereof. In some embodiments, the phospholipids comprise at least 40%, 50%, 60%, 70%, 80%, 90%, or 95% phosphatidylcholine by weight. In some embodiments, the phospholipids comprise greater than 80% phosphatidylcholine.

In some embodiments, the phospholipid is present in the pharmaceutical composition and/or in the amorphous solid dispersion in an amount of about 25 mg to about 200 mg. In some embodiments, the phospholipid is present in an amount of about 50 mg to 150 mg. In some embodiments, the phospholipids comprise 2.5%-20% of the total weight of the pharmaceutical composition. In some embodiments, the phospholipids comprise 5%-17% of the total weight of the pharmaceutical composition. In some embodiments, the phospholipids comprise greater than 80% phosphatidylcholine.

In some embodiments, the surfactant is a phospholipid. In some embodiments, the phospholipid is phosphatidylcholine. In some embodiments, the phospholipid is a mixture comprising phosphatidylcholine. In some embodiments, the surfactant is lecithin. In some embodiments, the lecithin is comprised of phosphatidylcholine. In some embodiments, the lecithin contains more than 25% of phosphatidylcholine. In some embodiments, the lecithin contains more than 80% of phosphatidylcholine. In some embodiments, the phosphatidylcholine is from egg origin. In some embodiments, the phosphatidylcholine is from or soybean origin.

In some embodiments, the surfactant is lecithin. The USP 40 definition of lecithin is “a complex mixture of acetone-insoluble phosphatides, which consist chiefly of phophatidylcholine, phosphatidylethanolamine, phosphatilinositol, and phosphatidic acid, present in conjunction with various amounts of other substances such as triglycerides, fatty acids, and carbohydrates, as separated from the crude vegetable oil source.” In some embodiments, lecithin is a mixture of phospholipids. Lecithin can be isolated from various sources including, but not limited to eggs, soybeans, milk, marine sources, rapeseed, cottonseed and sunflower. In some embodiments, the lecithin used in the disclosed amorphous solid dispersions and/or pharmaceutical compositions is isolated from egg yolk.

### Pharmaceutical Compositions

In certain embodiments, the invention provides pharmaceutical compositions comprising an amorphous solid dispersion that comprises an API. In some embodiments,
the amorphous solid dispersion comprises up to 99%, up to 90%, up to 85%, up to 80%, up to 75%, up to 70%, up to 65%, up to 60%, up to 55%, up to 50%, up to 45%, or up to 40% of the pharmaceutical composition by weight. In some embodiments,
the amorphous solid dispersion comprises from 10% to 90%, 20% to 90%, 30% to 90%, 40% to 90%, 50% to 90%, 60% to 90%, 70% to 90%, 80% to 90%, 30% to 80%, 40% to 80%, 50% to 80%, 60% to 80%, 20% to 90%, 20% to 80%, 20% to 70%, 20% to 60%, or 20% to 50% of the pharmaceutical composition by weight.

In some embodiments, the pharmaceutical composition comprises
the amorphous solid dispersion in an amount of about 50% to about 95% of a total weight of the pharmaceutical composition; microcrystalline cellulose in an amount of about 1% to about 12% of a total weight of the pharmaceutical composition; magnesium stearate in an amount of about 0.2% to about 5% of a total weight of the pharmaceutical composition; silica in an amount of about 0.2% to about 5% of a total weight of the pharmaceutical composition; and an organic acid in an amount of about 5% to about 20% of a total weight of the pharmaceutical composition.

The present pharmaceutical compositions can take the form of solutions, suspensions, emulsion, tablets, pills, pellets, capsules, capsules containing liquids, powders, suppositories, emulsions, suspensions, or any other form suitable for use. Preferred pharmaceutical compositions are formulated for oral delivery. In one embodiment, the pharmaceutically acceptable vehicle is a capsule. Capsules may be hard capsules or soft capsules, push-fit capsules made of gelatin, as well as soft, sealed capsules made of gelatin and a plasticizer (such as glycerol or sorbitol). In some embodiments, the capsule contains about 1000 mg of the pharmaceutical composition. In some embodiments, the capsule contains less than 1000 mg of the pharmaceutical composition. Capsules can be of any size. See, e.g., Remington's Pharmaceutical Sciences, page 1658-1659 (Alfonso Gennaro ed., Mack Publishing Company, Easton Pa., 18th ed., 1990), which is incorporated by reference.

The amorphous solid dispersions described herein can increase the dissolution rate of the API, e.g., as shown in FIG. 1. In some embodiments, about 80% or more of the API is dissolved in about 10 minutes or less. In some embodiments, about 70% or more of the API is dissolved in about 10 minutes or less. In some embodiments, about 60% or more of the API is dissolved in about 10 minutes or less. In some embodiments, about 50% or more of the API is dissolved in about 10 minutes or less. In some embodiments, about 80% or more of the API is dissolved in about 20 minutes or less. In some embodiments, about 70% or more of the API is dissolved in about 20 minutes or less. In some embodiments, about 60% or more of the API is dissolved in about 20 minutes or less. In some embodiments, about 50% or more of the API is dissolved in about 20 minutes or less. In some embodiments, about 80% or more of the API is dissolved in about 30 minutes or less. In some embodiments, about 70% or more of the API is dissolved in about 30 minutes or less. In some embodiments, about 60% or more of the API is dissolved in about 30 minutes or less. In some embodiments, about 50% or more of the API is dissolved in about 30 minutes or less. In some embodiments, about 80% or more of the API is dissolved in about 60 minutes or less. In some embodiments, about 70% or more of the API is dissolved in about 60 minutes or less. In some embodiments, about 60% or more of the API is dissolved in about 60 minutes or less. In some embodiments, about 50% or more of the API is dissolved in about 60 minutes or less.

### Methods of Treatment

In one aspect, disclosed herein are methods of treating a disease, wherein the method comprising administering a pharmaceutical composition or an amorphous solid dispersion described herein. Pharmaceutical compositions described herein can be administered for the treatment or prevention of diseases. When used to treat or prevent diseases or disorders, pharmaceutical compositions may be administered or applied singly, or in combination with other agents. Pharmaceutical compositions may also be administered or applied singly, in combination with other pharmaceutically active agents. Provided herein are methods of treatment and prophylaxis by administration to a subject in need of such treatment of a therapeutically effective amount of a pharmaceutical composition of the invention. The subject may be an animal, e.g., a mammal such as a human. In some embodiments, pharmaceutical compositions described herein are administered orally.

The pharmaceutical compositions described herein can be administered in prescribed regimens. In some embodiments, the pharmaceutical compositions are administered 3 times per day, twice per day, once per day, once every 2 days, once every 3 days, once every 4 days, once every 5 days, once every 6 days, once every week, twice every month, or once every month. In some embodiments, the dosage of API is 50 mg and is administered once a day. In some embodiments, the dosage of API is 75 mg and is administered once a day. In some embodiments, the dosage of API is 100 mg and is administered once a day. In some embodiments, the dosage of API is 125 mg and is administered once a day. In some embodiments, the dosage of API is 150 mg and is administered once a day. In some embodiments, the dosage of API is 200 mg and is administered once a day. In some embodiments, the dosage of API is 240 mg and is administered once a day.

The pharmaceutical compositions described herein can be administered with or without food. In some embodiments, the pharmaceutical composition is administered to the subject orally with food. In some embodiments, the pharmaceutical composition is administered to the subject orally without food. In some embodiments, the pharmaceutical composition is administered to the subject orally with food for 21 days followed by 7 days off treatment. In some embodiments, the pharmaceutical composition is administered to the subject orally with or without food for 21 days followed by 7 days off treatment. In some embodiments, the pharmaceutical composition is administered to the subject for a period of 1 to 7 weeks and followed by an off treatment of 1 to 2 weeks. In some embodiments, the pharmaceutical composition is administered to the subject for a period of one day to one year. In some embodiments, the pharmaceutical composition is administered approximately the same time each day.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein

### EXAMPLES

### Example 1: Materials and Methods

### Descriptions of the Material and Chemicals Used in the Present Invention

HPMCP-50 (HP-50)
HPMCAS-LF (AS-LF), HPMCAS-MF (AS-MF), HPMCAS-HF (AS-HF)
Salt of HPMCP-50: HPMCP-50-Na (HP-50-Na)
Polymer salt ionized by amines where cations are ammoniums: HPMCP-50-Amine (HP-50-Amine) eg. HP-50-TEA (TEA is the ammonium precursor)
HP-50-Sodium prolinate (HP-50-Proline-Na)
Proline used in the present invention is L-proline
DIPA (Diisopropylamine)
DIPEA (N,N-Diisopropylethylamine)
MIDEA (Methyldiethanolamine)
DEAE (Diethylethanolamine)
DMEA (Dimethylethanolamine)
Proline-Na (Sodium prolinate)
Proline-K (Potassium prolinate)
Proline-NH₄ (Ammonium prolinate)
DMG (Dimethylglycine)
DMG-Na (Dimethylglycine sodium salt)
PVAP-DMG-Na (PVAP was ionized by mixing PVAP and DMG-Na)

### Dissolution Performance Evaluation Experiment

Dissolution performance of ASDs in the present invention was evaluated by surface area normalized dissolution unless otherwise stated. All of the dissolution experiments were performed with 100 mg of material in 100 mL of pH 6.8 phosphate buffer (50 mM) at 37° C. unless otherwise specified. Error bars in the present invention indicate standard deviation, n=3

### Dissolution of Amorphous Solid Dispersions Using a Rotating Disk Apparatus

Surface area normalized dissolution was carried out using an intrinsic dissolution rate measurement assembly (Agilent, Santa Clara, CA). 100 mg of material was compressed at a pressure of 1500 psi with a hydraulic press (Carver Inc., Wabash, IN) in a circular intrinsic die of diameter 8 mm (corresponding to a surface area of 0.5 cm²), and the compression pressure was held for one minute. The die was then attached to a paddle rotating at 100 rpm unless otherwise stated.

### Concentration Analysis of Drug and Polymer

For the release studies, 0.2 mL and 0.03-0.2 mL of the dissolution medium were withdrawn for miconazole and polymer concentration analysis respectively and replaced with fresh buffer to maintain the volume at 100 mL. The typical time points taken were 10, 20, 30, 40, 50, 60, 80, 100 and 120 min. For miconazole, ritonavir, felodipine and clotrimazole, 0.2 mL of the sample was diluted by the addition of 0.4 mL of deionized water and 0.6 mL of methanol to obtain a clear solution, and the drug concentration was analyzed using a high-performance liquid chromatography (HPLC) system (1260 Infinity, Agilent, Santa Clara, CA). For the HPLC method of miconazole, a mobile phase of 80% (v/v) methanol in deionized water and 0.05% TFA (v/v) at a flow rate of 0.7 mL/min at 40° C. with an injection volume of 8 μL and an ultraviolet (UV) detection wavelength of 210 nm were used. For felodipine, a mobile phase of 80% (v/v) methanol in deionized water at a flow rate of 0.7 mL/min at 40° C. with an injection volume of 30 μL and an ultraviolet (UV) detection wavelength of 360 nm were used. For ritonavir, a mobile phase of 80% (v/v) methanol in deionized water at a flow rate of 0.7 mL/min at 40° C. with an injection volume of 20 μL and an ultraviolet (UV) detection wavelength of 210 nm were used. For clotrimazole, a mobile phase of 70% (v/v) methanol in deionized water and 0.1% TFA (v/v) at a flow rate of 0.75 mL/min at 40° C. with an injection volume of 8 μL and an ultraviolet (UV) detection wavelength of 210 nm were used. The separation column used was an Ascentis Express C18 (Sigma-Aldrich, St. Louis, MO) with dimensions of 10 cm×3.0 mm, 2.7 m particle size. HPMCP, HPMCAS and CAP quantification were carried out with colorimetric measurement (Reference: Li, N.; Ormes, J. D.; Taylor, L. S. Leaching of lopinavir amorphous solid dispersions in acidic media. Pharm. Res. 2016, 33, 1723-1735). 10 μL of phenol solution (4 g in 1 mL deionized water) was added to 0.4 mL of sample diluted to contain less than 100 μg/mL polymer and vortexed for 5 seconds. Then 1 mL of sulfuric acid was added and left to react for 60 min.

### HPMCP-50-X (HP-50-X) Structures and Code of New Polymers

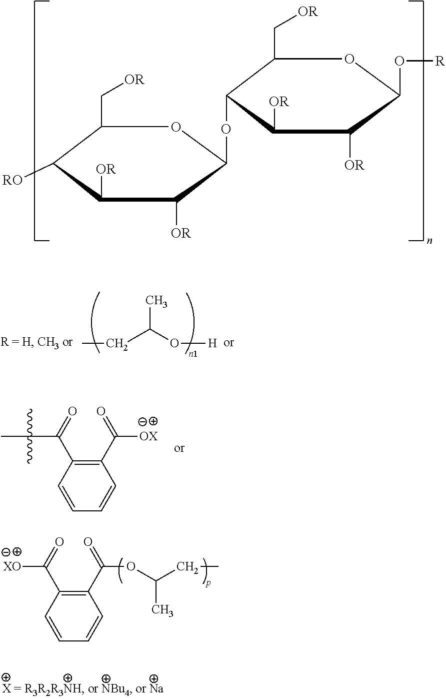

TABLE 1
HPMCP-50-X (HP-50-X)

| | Counterion | | | Counterion | Structure |
|---|---|---|---|---|---|
| Polymer | precursor | Structure of | Polymer | precursor (amine | of |
| code | (amine) | precursor | code | or ammonium) | precursor |
| PTHAM | Tris base | 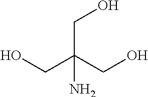 | PTEAA | Triethanolamine | 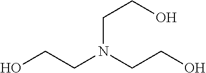 |
| PAMM | Ammediol | 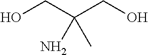 | PMDEA | MDEA | 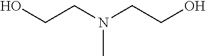 |
| PAMP | 2-Amino-2- methyl-1- propanol (AMP) | 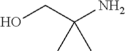 | PDEAE | DEAE | 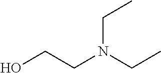 |
| PDIPA | DIPA | 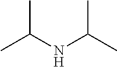 | PDMEA | 2- Dimethylaminoethanol (DMEA) | 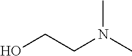 |
| PMP | Morpholine | 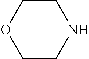 | PDIP | N,N- Diisopropylethylamine | 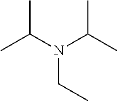 |
| PMEG | Meglumine | 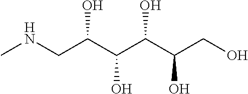 | PTEA | Triethylamine | 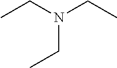 |
| PBTP | BIS-TRIS propane | 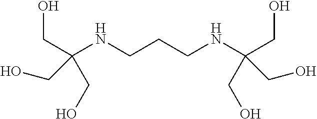 [SMILES: OCC(CO)(CO)NCCCNC(CO)(CO)CO] | PTBA | Tetrabutylammonium | ⊕ N(CH₂CH₂CH₂CH₃)₄ |
| PBTM | BIS-TRIS | 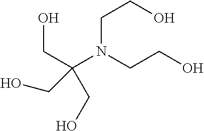 [SMILES: OCCN(CCO)C(CO)(CO)CO] | HP-Na | Sodium ion | Na⁺ |

### Example 2: Preparation of Neat Ionized Polymers

### Preparation of HPMCP-50-Na (HP-50-Na)

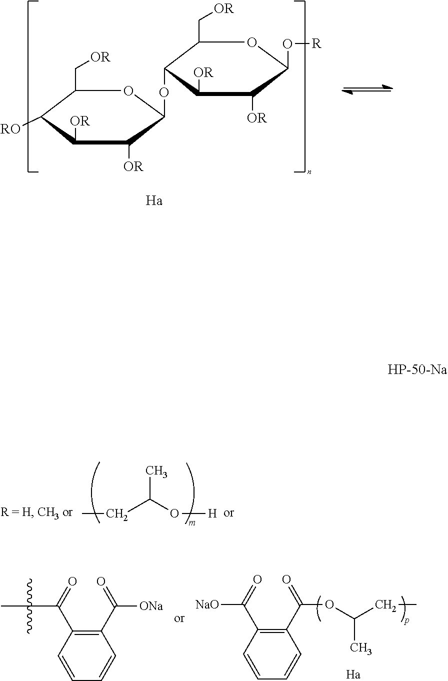

### Structure of HP-50-Na

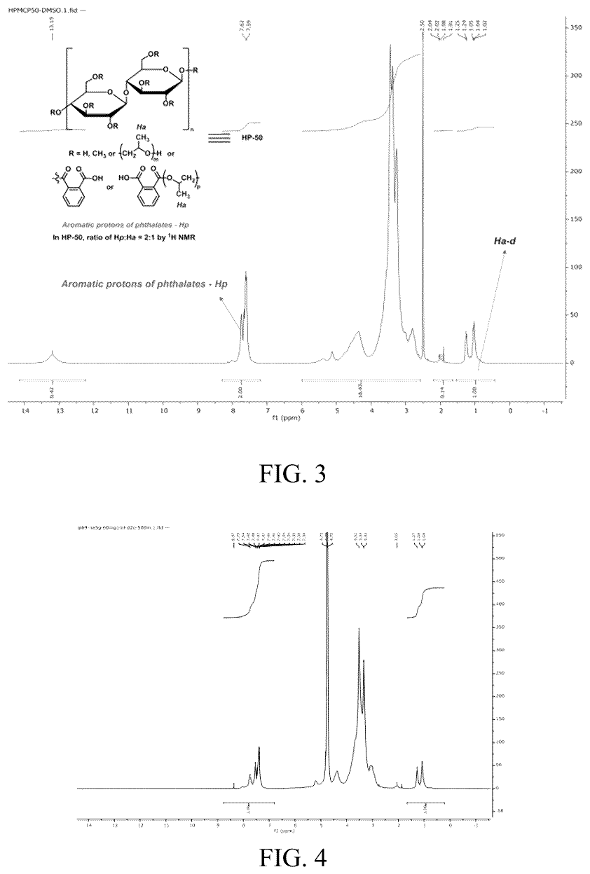

HPMCP-50 (1.0 g, containing 1.61 mmol COOH, 1.0 equiv.) was dissolved in DCM/MeOH (10 mL/15 mL). In another 20 mL vial, NaOMe (3.2 mL, 0.5 M in MeOH, 1.61 mmol, 1.0 equiv.) was added into a solution of HFIP (0.2 mL, 1.93 mmol, 1.2 equiv.) in MeOH (5 mL) and stirred for 30 min. Then the base solution was added into polymer solution within 5 min and stirred for 1 h. The mixture was concentrated under reduced pressure at 35° C. until a volume of 5-10 mL left, and H₂O (1 mL) was added, stirred for another 2 h. Acetone (80 mL) was then added to precipitate polymer followed by filtration and suspension in acetone (60 mL). After stirring for 10 h, the suspension was filtered, and the polymer salt was dried at 80° C. under reduced pressure in the presence of P₂O₅ for 5 h to give a fine powder. ¹H NMR in DMSO-d₆ indicated over 95% of phthalate moieties remained in polymer salt according to the ratio of Hp (7.28-7.71 ppm) to Ha (1.26-1.03 ppm) as 1.90 to 1.0 which is originally 2.0 to 1.0 in HPMCP-50. See FIGS. 2-3.

### HPMCP-50-Na in D₂O

To a 4 mL vial was added 60 mg HPMCP-50-Na and 1 mL D₂O, then the suspension was stirred for 30 min at room temperature to give a clear solution for ¹H NMR experiment. See FIG. 4.

### Fourier Transform Infrared (FTIR) Spectroscopy

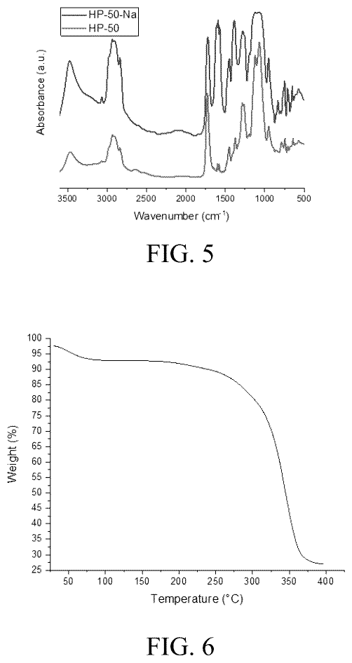

Thin films of neat HPMCP-50-Na and HPMCP-50 were prepared by spin-coating for collection of transmission IR spectra. The polymer was dissolved in MeOH/DCM (2:1 v/v) at a concentration of 50 mg/mL for spin-coating. 100 μL of solution was deposited onto a thallium bromoiodide (KRS-5) window (Harrick Scientific Corporation, Ossining, NY), then the substrate was firstly spun for 15 s at 50 rpm and another 50 s at 2500 rpm using a spin coater (Chemat Technology Inc., Northridge, CA). The spin-coating process was conducted in a humidity-controlled glovebox and then the substrate was dried in vacuum oven at room temperature for 24 h. The IR spectra were collected in transmission mode using a Bruker Vertex 70 FTIR spectrometer (Billerica, MA). 64 scans were collected for both the background and samples at a resolution of 4 cm⁻¹. The data were analyzed using OPUS software (version 7.2, Bruker, Billerica, MA). See FIGS. 5-6.

### Example 3: HPMCP-50-Na Preparation by Using NaHCO₃

HPMCP-50 (2.0 g) was placed in 100 mL flask and 20 mL deionized water was added. After stirring for 10 min, NaHCO₃ (271 mg) was added in two batches. The mixture was stirred until all polymer dissolved to give a clear solution. Then the solution was added dropwise into a cosolvents of acetone and acetonitrile under stirring. Precipitates were collected by filtration and dried in an oil bath at 80° C. under vacuum to remove residue solvents.

### Example 4: HPMCP-50-Na Preparation by Using NaHCO₃

HPMCP-50 (2.0 g) was placed in 100 mL flask and 20 mL deionized water was added. After stirring for 10 min, NaHCO₃ (271 mg) was added in two batches. The mixture was stirred until all polymer dissolved to give a clear solution. Water was evaporated via rotary evaporation at 35° C. using a Heidolph Hei-VAP Core rotary evaporator (Heidolph Instruments, Schwabach, Germany) coupled to a Ecodyst EcoChyll S cooler (Ecodyst, Apex, NC, USA) under reduced pressure to give polymer salt.

### Example 5: HPMCP-50-Na Preparation by Using NaHCO₃

HPMCP-50 (20.0 g) was placed in 1000 mL flask and deionized water (50 mL) was added. After stirring for 10 min, NaHCO₃ (1.353 g) solution in deionized water (40 mL) was added dropwise. The mixture was stirred until all polymer dissolved to give a clear solution. Then the solution was subjected to spray drying performed in a Mini Spray Dryer B-290 (Buchi, Switzerland) with inlet and outlet temperatures as 105° C. and 55° C. to give powder of HP-50-Na.

### Example 6: HPMCP-50-Na Preparation by Using Na₂CO₃

HPMCP-50 (2.0 g) was placed in 100 mL flask and 20 mL deionized water was added. After stirring for 10 min, Na₂CO₃ (171 mg) was added in two batches. The mixture was stirred until all polymer dissolved to give a clear solution. Then most of water was evaporated via rotary evaporation at 35° C. using a Heidolph Hei-VAP Core rotary evaporator (Heidolph Instruments, Schwabach, Germany) coupled to a Ecodyst EcoChyll S cooler (Ecodyst, Apex, NC, USA) under reduced pressure. 10 mL MeOH was added and incubated for 2 h followed by adding excess acetone under stirring. Precipitates were collected by filtration and heated in an oil bath at 80° C. under vacuum in the presence of P₂O₅ to remove residue solvents.

### Example 7: HPMCP-50-Na by Using NaOH

HPMCP-50 (2.0 g) was placed in 100 mL flask and 20 mL deionized water was added. After stirring for 10 min, NaOH (129 mg) in 10 mL deionized water was added dropwise. The mixture was stirred until all polymer dissolved to give a clear solution. Then most of water was evaporated via rotary evaporation at 35° C. using a Heidolph Hei-VAP Core rotary evaporator (Heidolph Instruments, Schwabach, Germany) coupled to a Ecodyst EcoChyll S cooler (Ecodyst, Apex, NC, USA) under reduced pressure. 10 mL MeOH was added and incubated for 2 h followed by adding excess acetone under stirring. Precipitates were collected by filtration and heated in an oil bath at 80° C. under vacuum in the presence of P₂O₅ to remove residue solvents.

### Example 8: HPMCP-50-K by Using KHCO₃

HPMCP-50 (10.0 g) was placed in 1000 mL flask and 50 mL deionized water was added. After stirring for 10 min, KHCO₃ (1.612 g) in 30 mL deionized water was added dropwise. The mixture was stirred until all polymer dissolved to give a clear solution. Then most of water was evaporated via rotary evaporation at 35° C. using a Heidolph Hei-VAP Core rotary evaporator (Heidolph Instruments, Schwabach, Germany) coupled to a Ecodyst EcoChyll S cooler (Ecodyst, Apex, NC, USA) under reduced pressure. 10 mL MeOH was added and incubated for 2 h followed by adding excess acetone under stirring. Precipitates were collected by filtration and heated in an oil bath at 80° C. under vacuum in the presence of P₂O₅ to remove residue solvents.

### Example 9: HPMCP-50-NH₄ by Using NH₄HCO₃

HPMCP-50 (2.0 g) was placed in 100 mL flask and 20 mL deionized water was added. After stirring for 10 min, NH₄HCO₃ (255 mg) was added in two batches. The mixture was stirred until all polymer dissolved to give a clear solution. Then most of water was evaporated via rotary evaporation at 35° C. using a Heidolph Hei-VAP Core rotary evaporator (Heidolph Instruments, Schwabach, Germany) coupled to a Ecodyst EcoChyll S cooler (Ecodyst, Apex, NC, USA) under reduced pressure. Obtained polymer was further dried in the presence of P₂O₅.

### Example 10: HPMCP-50-Tetrabutylammonium (PTBA) by Using Metathesis of Amino Sulfonic Acid

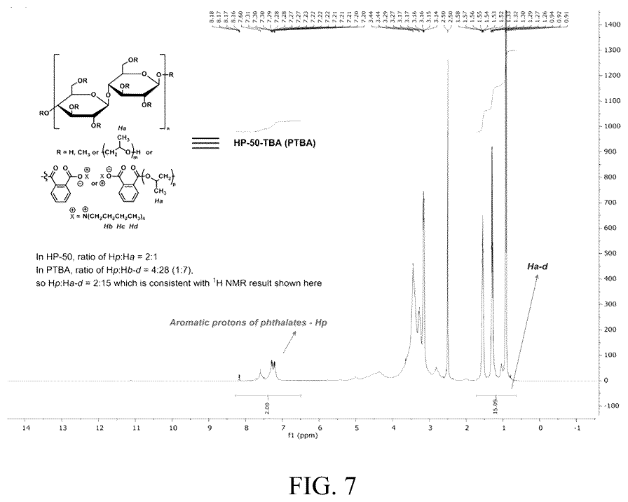

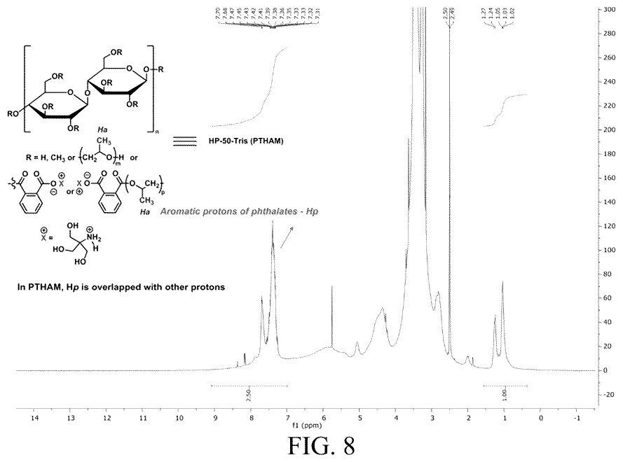

Taurine (201.5 mg, 1.61 mmol, 1.0 equiv.) and tetrabutylammonium hydroxide (1.61 mL, 1.0 M in methanol, 1.61 mmol, 1.0 equiv.) were mixed in MeOH (15 mL) and stirred at room temperature until all taurine dissolved to give a clear solution. Then this solution was added dropwise into a solution of HP-50 (1.0 g, containing 1.61 mmol COOH, 1.0 equiv.) in DCM/MeOH (15 mL/15 mL). After addition, the mixture was stirred overnight and DCM (30 mL) was added and stirred for another 30 min followed by filtration and concentration of the clear filtrates to give a slightly yellow polymer. The residue solvents were removed in a high vacuum oven at room temperature for 48 h. ¹H NMR in DMSO-d₆ indicated over 99% of phthalate moieties remained in PTBA as illustrated IN FIG. 7.

### Example 11: HPMCP-55-Na Preparation by Using NaHCO₃

HPMCP-55 (2.0 g) was placed in 100 mL flask and 20 mL deionized water was added. After stirring for 10 min, NaHCO₃ (349 mg) was added in two batches. The mixture was stirred until all polymer dissolved to give a clear solution. Then most of water was evaporated via rotary evaporation at 35° C. using a Heidolph Hei-VAP Core rotary evaporator (Heidolph Instruments, Schwabach, Germany) coupled to a Ecodyst EcoChyll S cooler (Ecodyst, Apex, NC, USA) under reduced pressure. 20 mL MeOH was added and incubated for 2 h followed by adding excess acetone under stirring. Precipitates were collected by filtration and heated in an oil bath at 80° C. under vacuum in the presence of P₂O₅ to remove residue solvents.

### Example 12: HPMCAS-LF-Na by Using NaHCO₃

HPMCAS-LF (2.0 g) was placed in 100 mL flask and 20 mL deionized water was added. After stirring for 10 min, NaHCO₃ (265 mg) was added in two batches. The mixture was stirred until all polymer dissolved to give a clear solution. Then most of water was evaporated via rotary evaporation at 35° C. using a Heidolph Hei-VAP Core rotary evaporator (Heidolph Instruments, Schwabach, Germany) coupled to a Ecodyst EcoChyll S cooler (Ecodyst, Apex, NC, USA) under reduced pressure. 10 mL MeOH was added and incubated for 2 h followed by adding excess acetone under stirring. Precipitates were collected by filtration and heated in an oil bath at 80° C. under vacuum in the presence of P₂O₅ to remove residue solvents.

### Example 13: HPMCAS-MF-Na by Using NaHCO₃

HPMCAS-MF (2.0 g) was placed in 100 mL flask and 20 mL deionized water was added. After stirring for 10 min, NaHCO₃ (200 mg) was added in two batches. The mixture was stirred until all polymer dissolved to give a clear solution. Then most of water was evaporated via rotary evaporation at 35° C. using a Heidolph Hei-VAP Core rotary evaporator (Heidolph Instruments, Schwabach, Germany) coupled to a Ecodyst EcoChyll S cooler (Ecodyst, Apex, NC, USA) under reduced pressure. 10 mL MeOH was added and incubated for 2 h followed by adding excess acetone under stirring. Precipitates were collected by filtration and heated in an oil bath at 80° C. under vacuum in the presence of P₂O₅ to remove residue solvents.

### Example 14: HPMCAS-HF—Na by Using NaOH

HPMCAS-HF (5.0 g) was placed in 250 mL flask and 30 mL deionized water was added. After stirring for 10 min, NaOH (119 mg) in 20 mL deionized water was added dropwise. The mixture was stirred until all polymer dissolved to give a clear solution. Then water was evaporated via rotary evaporation at 35° C. using a Heidolph Hei-VAP Core rotary evaporator (Heidolph Instruments, Schwabach, Germany) coupled to a Ecodyst EcoChyll S cooler (Ecodyst, Apex, NC, USA) under reduced pressure. Obtained polymer salt was further dried under vacuum in the presence of P₂O₅.

### Example 15: CAP-Na Preparation by Using NaHCO₃

CAP (5.0 g) was placed in 250 mL flask and 30 mL deionized water was added. After stirring for 10 min, NaHCO₃ (1.235 g) in deionized water (30 mL) was added dropwise. The mixture was stirred until all polymer dissolved to give a clear solution. Then most of water was evaporated via rotary evaporation at 35° C. using a Heidolph Hei-VAP Core rotary evaporator (Heidolph Instruments, Schwabach, Germany) coupled to a Ecodyst EcoChyll S cooler (Ecodyst, Apex, NC, USA) under reduced pressure. 30 mL MeOH was added and incubated for 3 h followed by adding excess acetone under stirring. Precipitates were collected by filtration and heated in an oil bath at 80° C. under vacuum in the presence of P₂O₅ to remove residue solvents.

### Example 16: CAP-Na Preparation by Using NaOH

CAP (5.0 g) was placed in 250 mL flask and 30 mL deionized water was added. After stirring for 10 min, NaOH (588 mg) in deionized water (30 mL) was added dropwise at 0° C. The mixture was stirred until all polymer dissolved to give a clear solution. Then most of water was evaporated via rotary evaporation at 35° C. using a Heidolph Hei-VAP Core rotary evaporator (Heidolph Instruments, Schwabach, Germany) coupled to a Ecodyst EcoChyll S cooler (Ecodyst, Apex, NC, USA) under reduced pressure. 40 mL MeOH was added and incubated for 3 h followed by adding excess acetone under stirring. Precipitates were collected by filtration and heated in an oil bath at 80° C. under vacuum in the presence of P₂O₅ to remove residue solvents.

### Example 17: PVAP-Na Preparation by Using NaHCO₃

PVAP (5.0 g) was placed in 250 mL flask and 30 mL deionized water was added. After stirring for 10 min, NaHCO₃ (1.647 g) in deionized water (20 mL) was added dropwise. The mixture was stirred until all polymer dissolved to give a clear solution. Then most of water was evaporated via rotary evaporation at 35° C. using a Heidolph Hei-VAP Core rotary evaporator (Heidolph Instruments, Schwabach, Germany) coupled to a Ecodyst EcoChyll S cooler (Ecodyst, Apex, NC, USA) under reduced pressure. The obtained polymer salt was further dried under vacuum in the presence of P₂O₅.

### Example 18: PTHAM Preparation

See FIG. 8.

### Example 19: PTEA Preparation

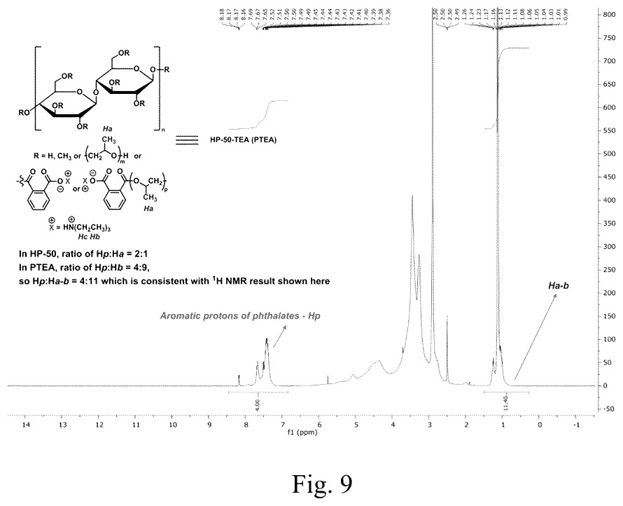

See FIG. 9.

### Example 20: PMP Preparation

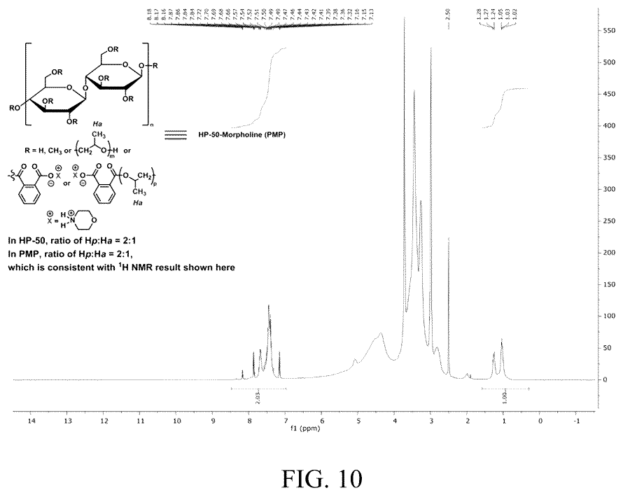

See FIG. 10.

### Example 21: PDIP Preparation

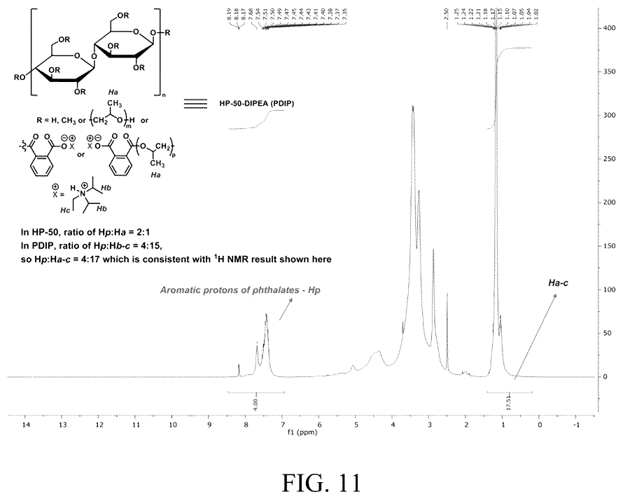

See FIG. 11.

### Example 22: PBTM Preparation

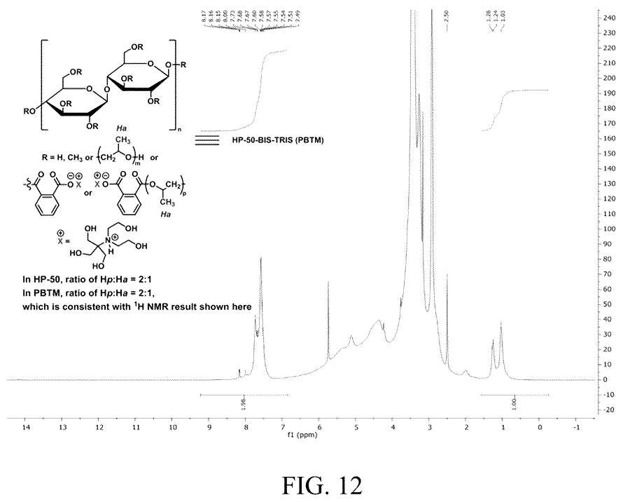

See FIG. 12.

### Example 23: PBTP Preparation

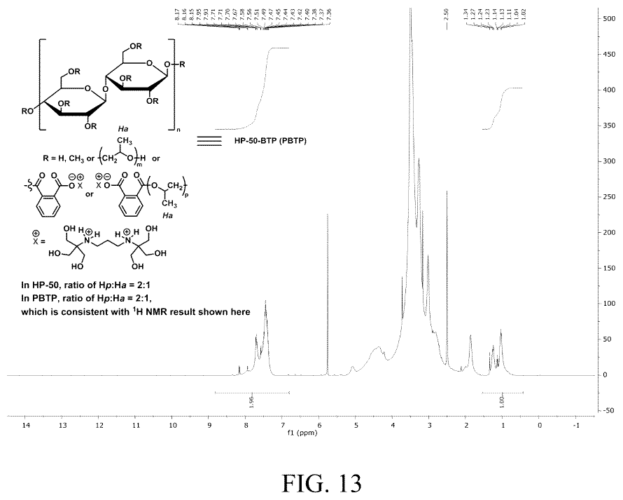

See FIG. 13.

### Example 24: PMEG Preparation

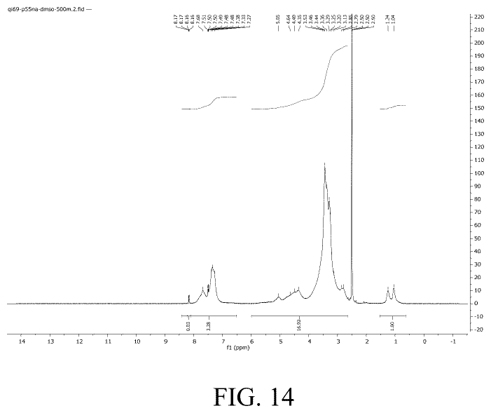

See FIG. 14.

### Example 25: PTEAA Preparation

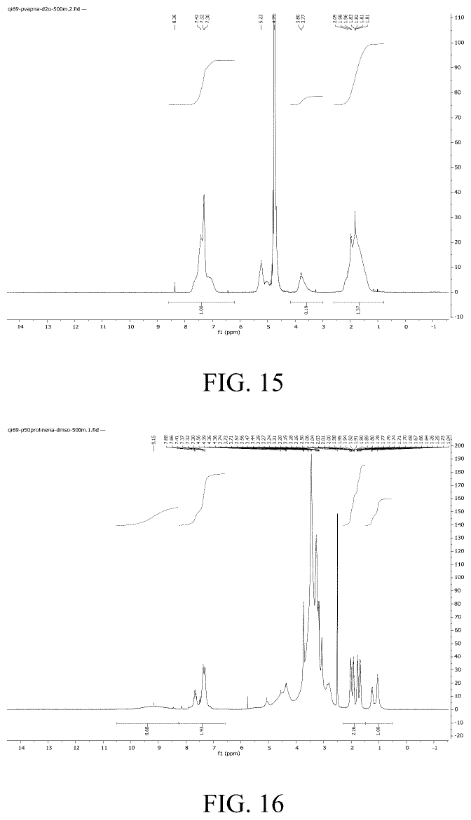

See FIG. 15.

### Example 26: AS—XF-Ammonium Preparation

See FIG. 16.

### Example 27: CAP-Ammonium

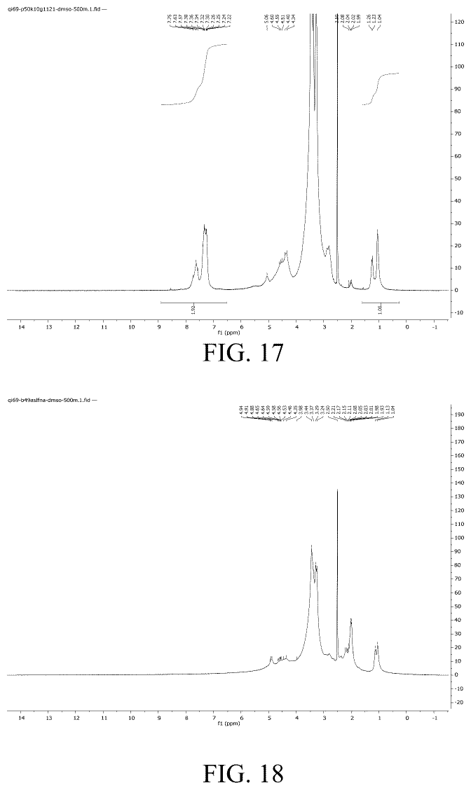

See FIG. 17.

### Example 28: PVAP-Ammonium

See FIG. 18.

### Example 29: Eudragit-L 100-ammonium

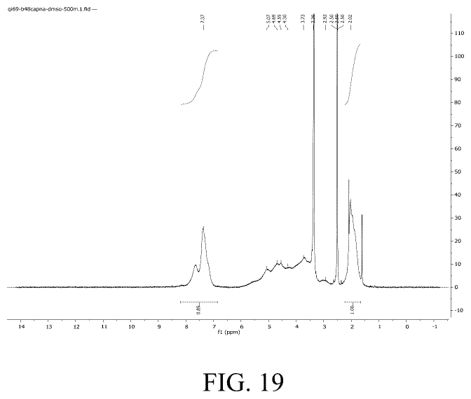

See FIG. 19.

### Example 30: Dissolution Performance of Ionized Polymers and Comparison with their Corresponding Unionized Polymers

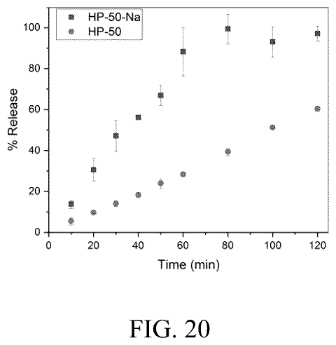

Release profiles of HP-50-Na and HP-50 over time in 50 mM pH 6.8 sodium phosphate buffer. HP-50-Na dissolves much faster than its protonated form (FIG. 20).

### Example 31: Normalized Dissolution Rate of Pre-Ionized Polymers is Over Two Times Faster than Protonated Polymers, Namely HP-50

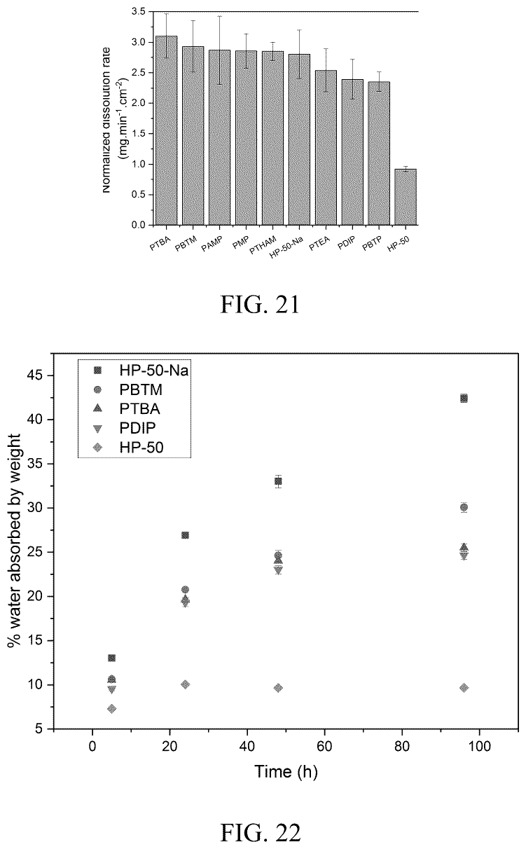

FIG. 21 shows that normalized dissolution rate of pre-ionized and protonated polymers in 50 mM pH 6.8 sodium phosphate buffer.

### Example 32: Hydration of Neat Polymers

HPMCP-50, HPMCP-50-Na and other neat polymers were cryomilled and dried in the presence of P₂O₅ under vacuum for 24 h. 200 mg of neat polymer powder was placed in a 4 mL glass vial and the powder was leveled. Then the open vial was stored at 100% RH at 37° C. The water sorption of neat polymers was measured gravimetrically at various time intervals for up to 96 hours with results summarized in FIG. 22.

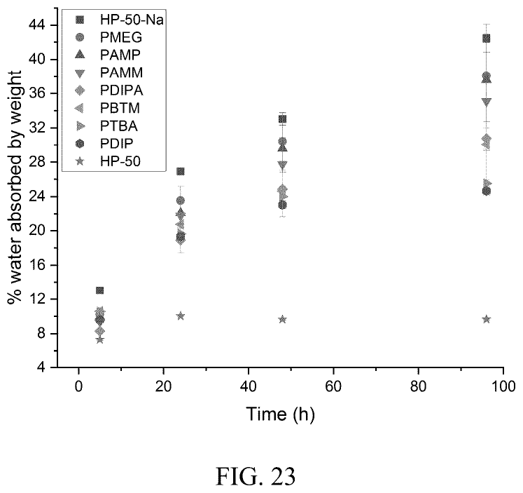

The Na polymer salt absorbed the most water, reaching more than 40% water. The protonated polymer had a much lower water content, while the three amine salts had intermediate water contents, with the salt with the more hydrophilic BIS-TRIS cation absorbing more water than the hydrophobic DIPEA salt and tetrabutylammonium salt, although the differences were more minimal than might be predicted from the chemical structure of the cations. A full summary of all tested neat polymers see FIG. 23.

### Example 33: HP-50-Na Showed Almost the Same Normalized Polymer Release Rate at Different Buffer Capacities while HP-50 Dissolves Much Slower at Lower Buffer Capacity

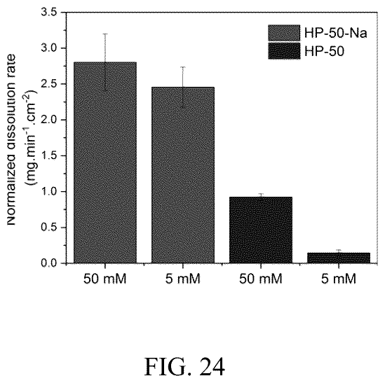

FIG. 24 shows normalized polymer release rate of HP-50-Na and HP-50 at different buffer capacities. 50 mM and 5 mM pH 6.8 sodium phosphate buffer were used here.

### Example 34: Effect of Ionization Percentage on Dissolution Rate of Neat Polymers (HP-50-BIS-TRIS)

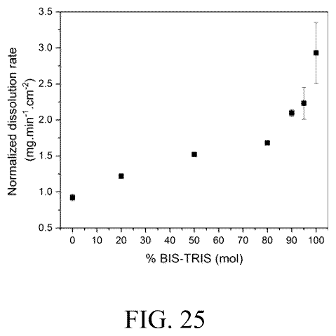

To further probe the relationship between polymer ionization and dissolution, a series of partially neutralized polymer salts was prepared with BIS-TRIS as the counterion. It is apparent that the dissolution rate decreases as the percent ionization is reduced from 100 to 20% ionized. The largest drop in dissolution rate occurs when the extent of ionization is reduced from 100 to 80% with a more modest decline thereafter. 50 mM pH 6.8 sodium phosphate buffer was used in all cases. See FIG. 25.

### Example 35: Methods of ASD Preparation

### HP-50-Na-Miconazole ASD at 20% Drug Loading

HP-50-Na (800 mg) and miconazole (200 mg) were added into a cosolvent of DCM/MeOH (40 mL, 1:1 v/v) under stirring to give a clear solution. The dissolved mixture was stirred for 30 min at room temperature followed by solvent evaporation at 50° C. using a Heidolph Hei-VAP Core rotary evaporator (Heidolph Instruments, Schwabach, Germany) coupled to an Ecodyst EcoChyllS cooler (Ecodyst, Apex, NC, USA) under reduced pressure. The obtained ASD was put in a high vacuum oven for 48 h at room temperature before it was pulverized with a 6750 Freezer/Mill cryogenic impact mill (SPEX SamplePrep, Metuchen, NJ, USA). The pulverized ASD powder was stored in a desiccator over calcium sulfate at room temperature overnight and used without further treatment.

### HP-50-TEA (PTEA)-Miconazole ASD at 20% Drug Loading

PTEA (800 mg) and miconazole (200 mg) were added into DCM (50 mL) under stirring to give a clear solution. The dissolved mixture was stirred for 30 min at room temperature followed by solvent evaporation at 40° C. using a Heidolph Hei-VAP Core rotary evaporator (Heidolph Instruments, Schwabach, Germany) coupled to an Ecodyst EcoChyllS cooler (Ecodyst, Apex, NC, USA) under reduced pressure. The obtained ASD was put in a high vacuum oven for 48 h at room temperature before it was pulverized with a 6750 Freezer/Mill cryogenic impact mill (SPEX SamplePrep, Metuchen, NJ, USA). The pulverized ASD powder was stored in a desiccator over calcium sulfate at room temperature overnight and used without further treatment.

### HP-50-Meglumine (PMEG)-Miconazole ASD at 20% Drug Loading

PMEG (800 mg) and miconazole (200 mg) were added into MeOH (40 mL) under stirring to give a clear solution. The dissolved mixture was stirred for 30 min at room temperature followed by solvent evaporation at 40° C. using a Heidolph Hei-VAP Core rotary evaporator (Heidolph Instruments, Schwabach, Germany) coupled to an Ecodyst EcoChyllS cooler (Ecodyst, Apex, NC, USA) under reduced pressure. The obtained ASD was put in a high vacuum oven for 48 h at room temperature before it was pulverized with a 6750 Freezer/Mill cryogenic impact mill (SPEX SamplePrep, Metuchen, NJ, USA). The pulverized ASD powder was stored in a desiccator over calcium sulfate at room temperature overnight and used without further treatment.

### HP-50-TEA (PTEA)-Miconazole ASD at 20% Drug Loading

Triethylamine (224 mg, 1.0 equiv to the phthalic acid in HP-50) was added into a solution of HP-50 (1.376 g) in MeOH/DCM (50 mL, 10:90, v/v) under stirring. After 60 min, miconazole (400 mg) was added and stirred for another 30 min before the solvent was evaporated.

### HP-50-TEA (PTEA)-Miconazole ASD at 20% Drug Loading in One-Pot

Triethylamine (224 mg, 1.0 equiv to the phthalic acid in HP-50) and miconazole (400 mg) was added into a solution of HP-50 (1.376 g) in MeOH/DCM (50 mL, 10:90, v/v) under stirring. After 60 min, the solvent was evaporated.

### HP-50-Meglumine (PMEG)-Miconazole ASD at 60% Drug Loading

Meglumine (80 mg, 1.0 equiv to the phthalic acid in HP-50) was added into a solution of HP-50 (254 mg) in MeOH/DCM (50 mL, 1:1, v/v) under stirring. After 60 min, miconazole (500 mg) was added and stirred for another 30 min before the solvent was evaporated.

### HPMCAS-MF-Ammediol-Miconazole ASD at 40% Drug Loading

Ammediol (100 mg, 1.0 equiv to the carboxylic acid group in HPMCAS-MF, 1.19 mmol/g was used in this case) was added into a solution of HPMCAS-MF (800 mg) in MeOH/DCM (50 mL, 1:1, v/v) under stirring. After 60 min, miconazole (600 mg) was added and stirred for another 30 min before the solvent was evaporated.

### CAP-Ammediol-Miconazole ASD at 40% Drug Loading

Ammediol (213 mg, 1.0 equiv to the phthalic acid group in CAP, 2.94 mmol/g was used in this case) was added into a solution of CAP (688 mg) in MeOH/DCM (50 mL, 1:1, v/v) under stirring. After 60 min, miconazole (600 mg) was added and stirred for another 30 min before the solvent was evaporated.

### HP-50-Sodium Prolinate-Miconazole ASD at 50% Drug Loading

Sodium prolinate (72 mg, 1.0 equiv to the phthalic acid in HP-50) was added into a solution of HP-50 (328 mg) in MeOH/DCM (50 mL, 10:90, v/v) under stirring. After 60 min, miconazole (400 mg) was added and stirred for another 30 min before the solvent was evaporated.

### HP-50-Sodium Prolinate-Miconazole ASD at 50% Drug Loading

HP-50-Na (339 mg), proline (61 mg, 1.0 equiv to the sodium in HP-50-Na) and miconazole (400 mg) were added into MeOH/DCM (50 mL, 50:50, v/v) under stirring. After 60 min, the solvent was evaporated.

### HP-50-Sodium Prolinate-Miconazole ASD at 30% Drug Loading

Proline (142 mg, 1.0 equiv to the phthalic acid in HP-50) was dissolved in MeOH (20 mL) under stirring. Then NaOMe (2.46 mL, 0.5 M in MeOH) was added and stirred for 60 min to afford sodium prolinate solution, which was added dropwise into a solution of HP-50 (765 mg) in MeOH/DCM (40 mL, 1:3, v/v) under stirring. After 60 min, miconazole (400 mg) was added and stirred for another 30 min before the solvent was evaporated.

### HP-50-Potassium Prolinate-Miconazole ASD at 65% Drug Loading

Potassium prolinate (96 mg, 1.0 equiv to the phthalic acid in HP-50) was added into a solution of HP-50 (389 mg) in MeOH/DCM (50 mL, 10:90, v/v) under stirring. After 60 min, miconazole (900 mg) was added and stirred for another 30 min before the solvent was evaporated.

### HP-50-Sodium Prolinate-Miconazole ASD at 20% Drug Loading in One-Pot

Sodium prolinate (289 mg, 1.0 equiv to the phthalic acid in HP-50) and miconazole (400 mg) was added into a solution of HP-50 (1311 mg) in MeOH/DCM (50 mL, 10:90, v/v) under stirring. After 60 min, the solvent was evaporated.

### HP-50-Sodium Prolinate-Felodipine ASD at 30% Drug Loading

Proline (142 mg, 1.0 equiv to the phthalic acid in HP-50) was dissolved in MeOH (20 mL) under stirring. Then NaOMe (2.46 mL, 0.5 M in MeOH) was added and stirred for 60 min to afford sodium prolinate solution, which was added dropwise into a solution of HP-50 (765 mg) in MeOH/DCM (40 mL, 1:3, v/v) under stirring. After 60 min, felodipine (400 mg) was added and stirred for another 30 min before the solvent was evaporated.

### HP-50-Betaine-Felodipine ASD at 30% Drug Loading

Betaine (44 mg, 1.0 equiv to the phthalic acid in HP-50) was added into a solution of HP-50 (236 mg) in MeOH/DCM (20 mL, 20:80, v/v) under stirring. After 60 min, miconazole (120 mg) was added and stirred for another 30 min before the solvent was evaporated.

PVAP-Miconazole ASD at 20% Drug Loading

Miconazole (100 mg) was added into a solution of PVAP (400 mg) in MeOH (50 mL) under stirring. After 30 min, solvent was evaporated.

### PVAP-DMEA-Miconazole ASD at 50% Drug Loading

DMEA (39 mg, 1.0 equiv to the carboxylic acid group in PVAP, 3.92 mmol/g was used in this case) was added into a solution of PVAP (111 mg) in MeOH (20 mL) under stirring. After 60 min, miconazole (150 mg) was added and stirred for another 30 min before the solvent was evaporated.

### PVAP-TEA-Miconazole ASD at 50% Drug Loading

TEA (43 mg, 1.0 equiv to the carboxylic acid group in PVAP, 3.92 mmol/g was used in this case) was added into a solution of PVAP (107 mg) in MeOH (20 mL) under stirring. After 60 min, miconazole (150 mg) was added and stirred for another 30 min before the solvent was evaporated.

### PVAP-TEA-Miconazole ASD at 50% Drug Loading in One-Pot

TEA (43 mg, 1.0 equiv to the carboxylic acid group in PVAP, 3.92 mmol/g was used in this case) and miconazole (150 mg) was added into a solution of PVAP (107 mg) in MeOH (20 mL) under stirring. After 60 min, the solvent was evaporated.

### PVAP-DMEA-Felodipine ASD at 30% Drug Loading

DMEA (91 mg, 1.0 equiv to the carboxylic acid group in PVAP, 3.92 mmol/g was used in this case) was added into a solution of PVAP (259 mg) in MeOH (50 mL) under stirring. After 60 min, felodipine (150 mg) was added and stirred for another 30 min before the solvent was evaporated.

### PVAP-TEA-Felodipine ASD at 30% Drug Loading

TEA (99 mg, 1.0 equiv to the carboxylic acid group in PVAP, 3.92 mmol/g was used in this case) was added into a solution of PVAP (251 mg) in MeOH (50 mL) under stirring. After 60 min, felodipine (150 mg) was added and stirred for another 30 min before the solvent was evaporated.

### PVAP-DMEA-Ledipasvir ASD at 10% Drug Loading

DMEA (93 mg, 1.0 equiv to the carboxylic acid group in PVAP, 3.92 mmol/g was used in this case) was added into a solution of PVAP (267 mg) in MeOH (30 mL) under stirring. After 60 min, ledipasvir (40 mg) was added and stirred for another 30 min before the solvent was evaporated.

### PVAP-Na-Lopinavir ASD at 40% Drug Loading

Lopinavir (200 mg) was added into a solution of PVAP-Na (300 mg) in MeOH (50 mL) under stirring. After 30 min, solvent was evaporated.

### PVAP-DMEA-Lopinavir ASD at 50% Drug Loading

DMEA (52 mg, 1.0 equiv to the carboxylic acid group in PVAP, 3.92 mmol/g was used in this case) was added into a solution of PVAP (148 mg) in MeOH (40 mL) under stirring. After 60 min, lopinavir (200 mg) was added and stirred for another 30 min before the solvent was evaporated.

### PVAP-DMG-Na-Miconazole at 50% Drug Loading

DMG (Dimethylglycine) (27 mg, 1.0 equiv to the carboxylic acid group in PVAP, 3.92 mmol/g was used in this case) was dissolved in MeOH (10 mL) under stirring. Then NaOMe (0.53 mL, 0.5 M in MeOH) was added and stirred for 60 min to afford dimethylglycine sodium solution, which was added dropwise into a solution of PVAP (67 mg) in MeOH (30 mL) under stirring. After 60 min, miconazole (100 mg) was added and stirred for another 30 min before the solvent was evaporated.

### PVAP-DMG-Na-Lopinavir (40%)-Ritonavir (10%) at 50% Total Drug Loading

DMG (Dimethylglycine) (68 mg, 1.0 equiv to the carboxylic acid group in PVAP, 3.92 mmol/g was used in this case) was dissolved in MeOH (10 mL) under stirring. Then NaOMe (1.32 mL, 0.5 M in MeOH) was added and stirred for 60 min to afford dimethylglycine sodium solution, which was added dropwise into a solution of PVAP (168 mg) in MeOH (20 mL) under stirring. After 60 min, lopinavir (200 mg) and ritonavir (50 mg) were added and stirred for another 30 min before the solvent was evaporated.

### PVAP-DMG-Na-Lopinavir at 50% Drug Loading

DMG (Dimethylglycine) (81 mg, 1.0 equiv to the carboxylic acid group in PVAP, 3.92 mmol/g was used in this case) was dissolved in MeOH (10 mL) under stirring. Then NaOMe (1.58 mL, 0.5 M in MeOH) was added and stirred for 60 min to afford dimethylglycine sodium solution, which was added dropwise into a solution of PVAP (201 mg) in MeOH (30 mL) under stirring. After 60 min, lopinavir (300 mg) was added and stirred for another 30 min before the solvent was evaporated.

### PVAP-DMG-Na-Ritonavir at 50% Drug Loading

DMG (Dimethylglycine) (68 mg, 1.0 equiv to the carboxylic acid group in PVAP, 3.92 mmol/g was used in this case) was dissolved in MeOH (10 mL) under stirring. Then NaOMe (1.32 mL, 0.5 M in MeOH) was added and stirred for 60 min to afford dimethylglycine sodium solution, which was added dropwise into a solution of PVAP (168 mg) in MeOH (30 mL) under stirring. After 60 min, ritonavir (250 mg) was added and stirred for another 30 min before the solvent was evaporated.

### PVAP-DMG-Na-Ledipasvir at 40% Drug Loading

DMG (Dimethylglycine) (49 mg, 1.0 equiv to the carboxylic acid group in PVAP, 3.92 mmol/g was used in this case) was dissolved in MeOH (10 mL) under stirring. Then NaOMe (0.95 mL, 0.5 M in MeOH) was added and stirred for 60 min to afford dimethylglycine sodium solution, which was added dropwise into a solution of PVAP (121 mg) in MeOH (30 mL) under stirring. After 60 min, ledipasvir (120 mg) was added and stirred for another 30 min before the solvent was evaporated.

### CAP-DMG-Na-Lopinavir (32%)-Ritonavir (8%) ASD at 40% Total Drug Loading

DMG-Na (Dimethylglycine sodium) (97 mg, 1.0 equiv to the carboxylic acid group in CAP, 2.94 mmol/g was used in this case) was added into a solution of CAP (263 mg) in MeOH (50 mL) under stirring. After 60 min, lopinavir (192 mg) and ritonavir (48 mg) were added and stirred for another 30 min before the solvent was evaporated.

### Example 36: Dissolution Performance of Ionized Polymer ASDs and Comparison with their Corresponding Unionized Polymer ASDs

### Release Profiles of Miconazole-HPMCP-50-X (X═H, Na, TEA) ASDs

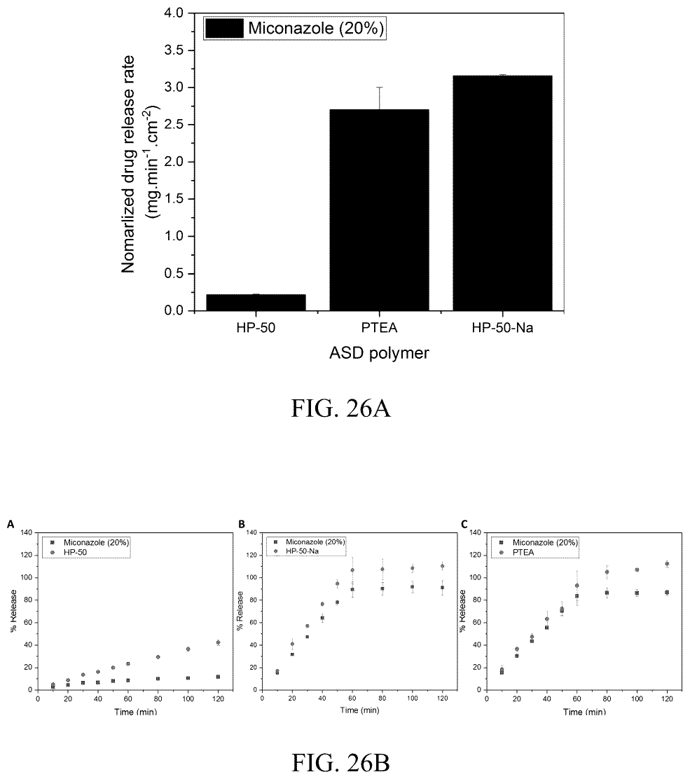

The impact of polymer salts with two different cations on the release behavior of miconazole was studied by surface normalized dissolution of ASD compacts at a 20% drug loading. Release from HPMCP-50 ASD was slow and incomplete, where the polymer released faster than the drug (incongruent release of components). Only 10% of the drug dose was released after 120 min. The HPMCP-50-Na ASD dissolved very quickly and exceeded the drug amorphous solubility (˜5 μg/mL) with the formation of a drug-rich phase leading to a cloudy dissolution medium. Close to 90% drug release was observed after 60 min, whereby the polymer released at the same normalized rate as the drug. The drug release rate from the HP-50-Na ASD is 14 times faster than the drug release from the HPMCP-50 ASD. A similar outcome was observed for the HPMCP-50-TEA (PTEA) ASD. See FIGS. 26A-B.

Drug Release Profiles of HP-50-Na-Miconazole ASD at Different Drug Loadings with Rapid Dissolution Rate

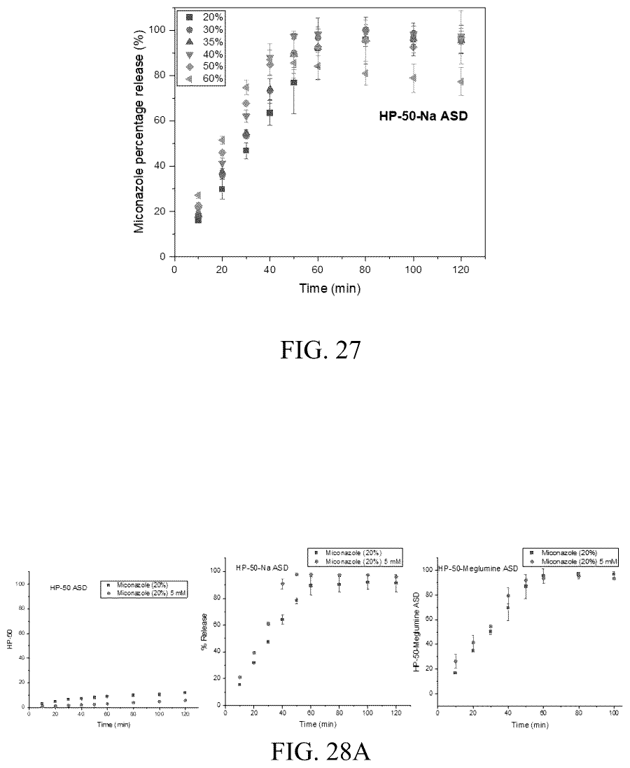

See FIG. 27 showing drug release percentage of 60% drug loading ASD at 30 min is over 60%.

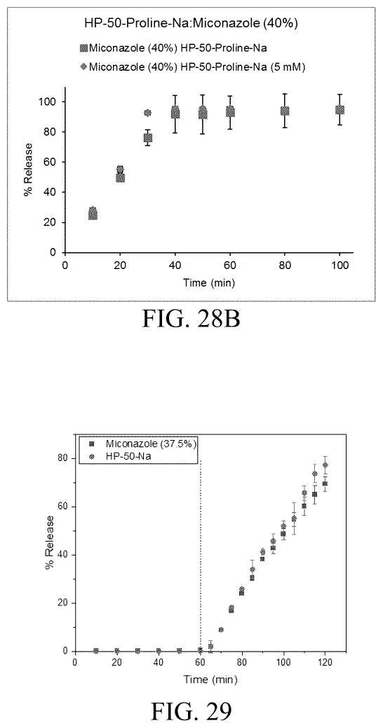

Miconazole ASDs with HP-50-Na, HP-50-Meglumine and HP-50-Proline-Na were able to maintain a rapid dissolution rate, irrespective of the buffer capacity of the medium at the same or twice higher drug loading, whereas the protonated polymer, HP-50, ASD showed greatly diminished dissolution as medium buffer capacity decreased toward physiological gastrointestinal tract values. See FIGS. 28A-B.

### Release Profile of HP-50-Na-Miconazole ASD in pH 1.6 and pH 6.8 Phosphate Buffer (50 mM). 150 mg Material was Used in the Experiment

95 mL buffer in beaker was warmed up to 37° C. with magnetic stirring at 100 rpm. 150 mg ASD powder with 100 mg Eudragit S 100 on top was compressed by 1500 psi then was immersed in buffer. 0.2 mL buffer was withdrawn every 10 min, to which was added 0.2 mL water and 0.4 mL MeOH (diluted six times) for HPLC analysis. 0.03-0.2 mL of the dissolution medium were withdrawn for polymer concentration analysis and replaced with fresh buffer to maintain the volume at 95 mL. 60 min later, 2.55 mL stock solution of NaOH was added in one portion to adjust pH up to 6.8. 0.03-0.2 mL buffer were withdrawn every 5 min for drug and polymer concentration analysis. The total amount of dissolution medium was maintained at 97.55 mL by replacing with fresh pH 6.8 phosphate buffer. See FIG. 29.

Powder Dissolution of HP-50-Na ASD at 20% Drug Loading with Drug Release in 50 mM pH 1.6 Sodium Phosphate Buffer

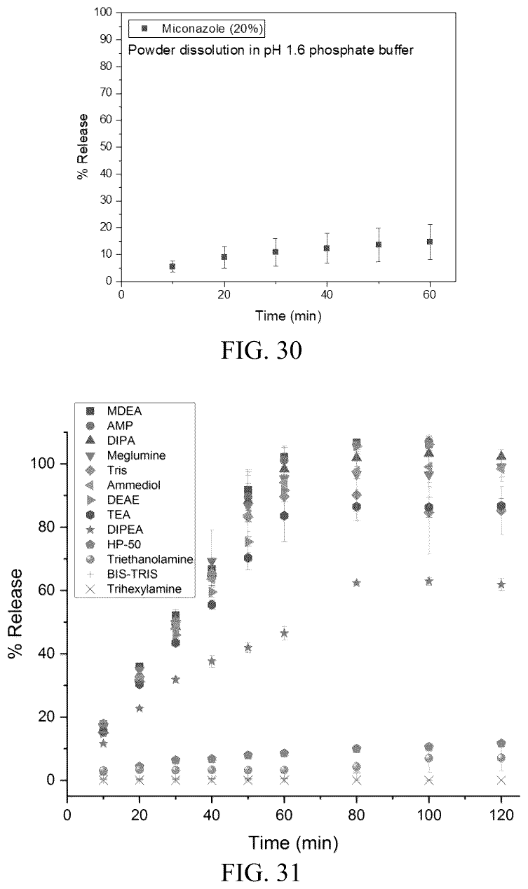

50 mL buffer in beaker was warmed up to 37° C. with magnetic stirring at 100 rpm. 50 mg ASD powder was dropped in buffer. 1 mL medium was withdrawn and filtered through glass fiber filter (0.2 um), then 0.5 mL of filtrate was added 0.5 mL MeOH (diluted two times) for HPLC analysis of drug. The total amount of dissolution medium was maintained at 50 mL by replacing with fresh buffer. See FIG. 30.

### Example 37A: Dissolution of HP-50-Ammonium-Miconazole ASD

Release profiles of HP-50-Ammonium-Miconazole ASDs at 20% drug loading. HP-50-Na-Miconazole ASD (20% DL) was used here as reference. See FIG. 31.

Summarized miconazole release percentage of HP-50-Ammonium-Miconazole ASDs at 30 min and 60 min respectively.

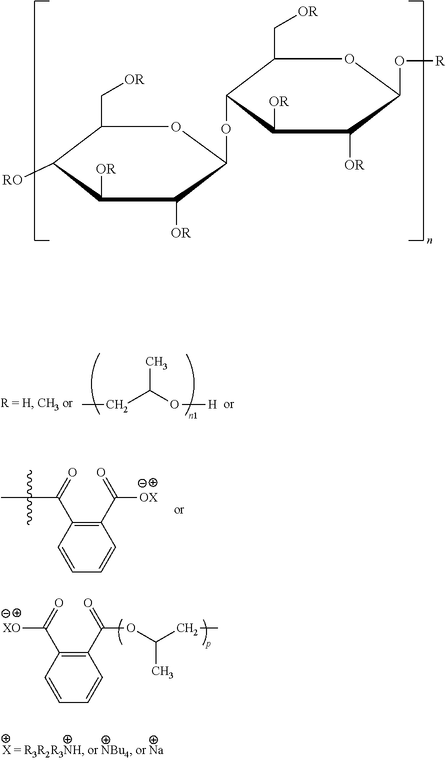

TABLE 2
HPMCP-50-X (HP-50-X)

| Ammonium | | | Drug release | Drug release |
|---|---|---|---|---|
| counterion | Structure of | Drug | percentage at | percentage at |
| precursor | precursor | loading | 30 min (%) | 60 min (%) |
| Tris base | 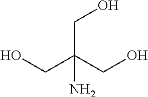 | 20% | 50 | 90 |
| Ammediol | 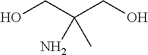 | 60% | >55 | >70 |
| 2-Amino-2-methyl-1- propanol (AMP) | 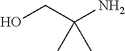 | 50% | >60 | >80 |
| DIPA | 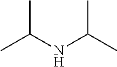 | 60% | >40 | 60 |
| Meglumine | 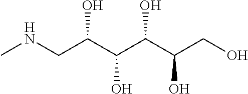 | 60% | >60 | >80 |
| MDEA | 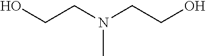 | 60% | 40 | 56 |
| DEAE | 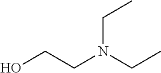 | 60% | >40 | 59 |
| 2-Dimethylaminoethanol (DMEA) | 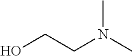 | 50% | 80 | >80 |
| Triethylamine (TEA) | 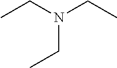 | 20% | >40 | >85 |
| N,N-Diisopropylethylamine (DIPEA) | 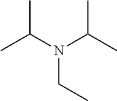 | 20% | >30 | >40 |

### Example 37B3: Dissolution of HP-50-Proline-Na-Miconazole ASD (HP-50-Proline-Na-Miconazole is Made as Above Mentioned)

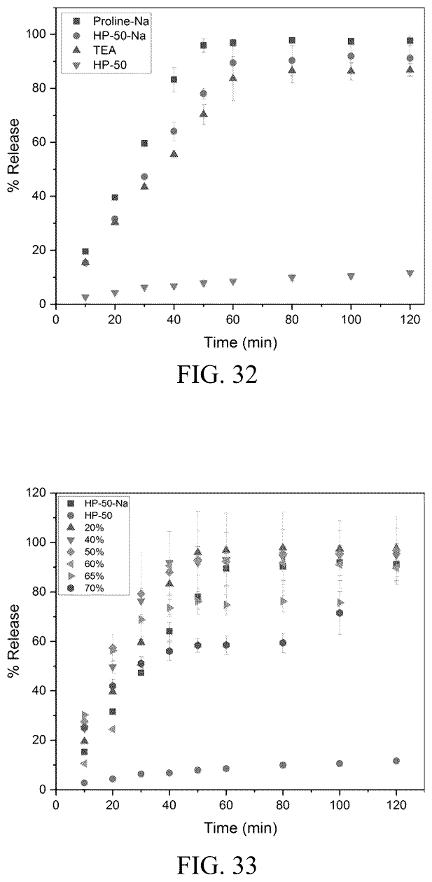

Dissolution of HP-50-Proline-Na-Miconazole ASD at 20% drug loading. HIP-50-TEA-Miconazole, TIP-50-Na-Miconazole and TIP-50-Miconazole ASDs at 20% drug loading were used here as references. See FIG. 32.

Dissolution of HP-50-Proline-Na-Miconazole ASD at different drug loadings. HP-50-Na-Miconazole and TIP-50-Miconazole ASDs at 20% drug loading were used here as references. Miconazole release percentage of HP-50-Proline-Na-Miconazole ASDs (up to 60% drug loading) at 50 min was over 80%. See FIG. 33.

### Example 38: Dissolution of HP-50-Ammonium-Felodipine ASD

Summarized felodipine release percentage of HP-50-Ammonium-Felodipine ASDs at 100 min. See Table 3.

TABLE 3

| Ammonium counterion | Structure of | Drug | Drug release percentage |
|---|---|---|---|
| precursor | precursor | loading | at 100 min (%) |
| Tris base | 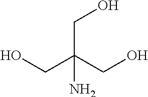 | 30% | 70 |
| Ammediol | 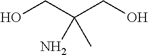 | 30% | >80 |
| Meglumine | 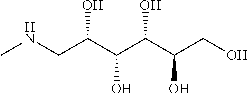 | 30% | >60 |
| Triethanolamine | 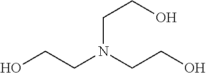 | 30% | 80 |
| MDEA | 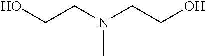 | 60% | >70 |

### Example 39: Dissolution of HP-50-Ammonium-Ritonavir ASDs

Summarized ritonavir release percentage of HP-50-Ammonium-Ritonavir ASDs at 50 min. See Table 4.

TABLE 4

| Ammonium | | | Drug release | | Drug release |
|---|---|---|---|---|---|
| counterion | Structure of | Drug | percentage at | Drug | percentage at |
| precursor | precursor | loading | 50 min (%) | loading | 50 min (%) |
| Tris base | 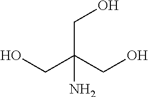 | 40% | >50 | — | — |
| Ammediol | 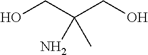 | 40% | >60 | 35% | >80 |
| Meglumine | 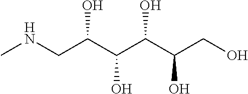 | 40% | >70 | 35% | 90 |
| MDEA | 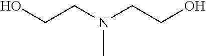 | 30% | 80 | 35% | 80 |
| 2-Dimethylaminoethanol (DMEA) | 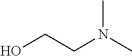 | 40% | 40 | 35% | >80 |

### Example 40: Dissolution of HP-50-Proline-Na-Ritonavir ASD (HP-50-Proline-Na-Ritonavir ASD is Made as Above Mentioned)

Ritonavir release percentage of HP-50-Proline-Na-Ritonavir ASDs (35% drug loading) at 50 min was over 85%.

### Example 41: Dissolution of HP-50-Proline-Na-Clotrimazole ASD (HP-50-Proline-Na-Clotrimazole ASD is Made as Above Mentioned)

Clotrimazole release percentage of HP-50-Proline-Na-Clotrimazole ASDs (40% drug loading) at 100 min was over 70%.

### Example 42: Dissolution of HP-50-Proline-Na-Felodipine ASD (HP-50-Proline-Na-Felodipine ASD is Made as Above Mentioned)

Felodipine release percentage of HP-50-Proline-Na-Felodipine ASDs (20% drug loading) at 80 min was over 80%.

### Example 43: Dissolution of HP-50-Proline-K-Miconazole ASD

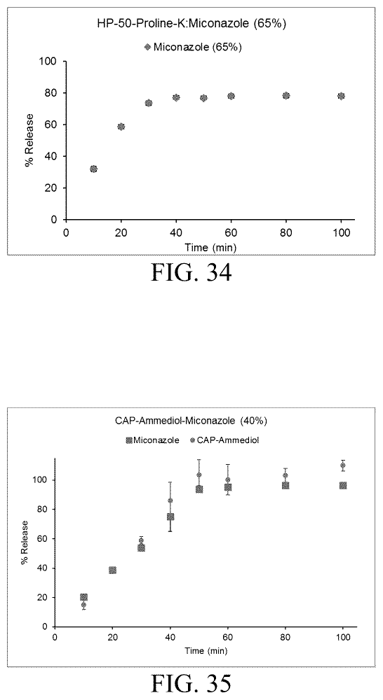

Miconazole release percentage of HP-50-Proline-K-Miconazole ASDs (65% drug loading) at 40 min was over 60%. See FIG. 34.

### Example 44: Dissolution of AS-LF-Meglumine-Miconazole ASD

Miconazole release percentage of AS-LF-Meglumine-Miconazole ASD (60% drug loading) at 100 min was over 80%.

### Example 45: Dissolution of AS-LF-Ammediol-Miconazole ASD

Miconazole release percentage of AS-LF-Meglumine-Miconazole ASD (40% drug loading) at 100 min was over 40%.

### Example 46: Dissolution of CAP-Ammediol-Miconazole ASD

FIG. 35 shows dissolution of CAP-Ammediol-Miconazole ASD (40% drug loading) and miconzaole release at 50 min is over 80%.

### Example 47: Dissolution of HP-50-Betaine-Felodipine ASD

Felodipine release percentage of HP-50-Betaine-Felodipine ASD (30% drug loading) at 80 min was over 40%.

### Example 48: Dissolution of HP-50-Felodipine ASD

Felodipine release percentage of HP-50-Felodipine ASD (30% drug loading) at 80 min was less than 30%.

### Example 49: Dissolution of PVAP-Miconazole ASD

Miconazole release percentage of PVAP-Miconazole ASD (20% drug loading) at 80 min was less than 10%.

### Example 50: Dissolution of PVAP-Ammonium-Miconazole ASDs

Summarized miconazole release percentage of PVAP-Ammonium-Miconazole ASDs at 20 min. See Table 5.

TABLE 5

| Ammonium | Structure | | Drug release |
|---|---|---|---|
| counterion | of | Drug | percentage at |
| precursor | precursor | loading | 20 min (%) |
| Triethylamine | 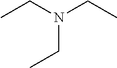 | 50% | >70 |
| 2-Dimethylaminoethanol (DMEA) | 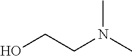 | 50% | >70 |

### Example 51: Dissolution of PVAP-DMG-Na-Miconazole ASD

Miconazole release percentage of PVAP-DMG-Na-Miconazole ASDs (50% drug loading) at 20 min was over 70%.

### Example 52: Dissolution of PVAP-Ammonium-Felodipine ASDs

Summarized felodipine release percentage of PVAP-Ammonium-Felodipine ASDs at 60 min. See Table 6.

TABLE 6

| Ammonium | Structure | | Drug release |
|---|---|---|---|
| counterion | of | Drug | percentage at |
| precursor | precursor | loading | 60 min (%) |
| Triethylamine | 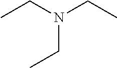 | 30% | >60 |
| 2-Dimethylaminoethanol (DMEA) | 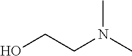 | 30% | >80 |

### Example 53: Dissolution of PVAP-DMG-Na-Felodipine ASD

Felodipine release percentage of PVAP-DMG-Na-Felodipine ASDs (30% drug loading) at 60 min was over 90%.

### Example 54: Dissolution of PVAP-Ammonium-Ledipasvir ASDs

Summarized drug release percentage of PVAP-Ammonium-Ledipasvir ASDs at 80 min. See Table 7.

TABLE 7

| Ammonium | Structure | | Drug release |
|---|---|---|---|
| counterion | of | Drug | percentage at |
| precursor | precursor | loading | 80 min (%) |
| Triethylamine | 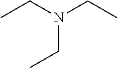 | 10% | >85 |
| 2-Dimethylaminoethanol (DMEA) | 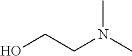 | 10% | >70 |

### Example 55: Dissolution of PVAP-DMG-Na-Ledipasvir ASD

Ledipasvir release percentage of PVAP-DMG-Na-Ledipasvir ASD (10% drug loading) at 80 min was over 70%.

### Example 56: Dissolution of PVAP-DMG-Na-Ritonavir ASD

Ritonavir release percentage of PVAP-DMG-Na-Ritonavir ASD (50% drug loading) at 60 min was over 95%.

### Example 57: Dissolution of PVAP-DMG-Na-Lopinavir ASD

Lopinavir release percentage of PVAP-DMG-Na-Lopinavir ASD (50% drug loading) at 60 min was over 95%.

### Example 58: Dissolution of PVAP-DMG-Na-Lopinavir (40%)-Ritonavir (10%) ASD

Drug release percentages of PVAP-DMG-Na-Lopinavir (40%)-Ritonavir (10%) ASD (50% drug loading in total) at 80 min were both over 75%.

### Example 59: Dissolution of PVAP-Na-Lopinavir ASD

Lopinavir release percentage of PVAP-Na-Lopinavir ASD (40% drug loading) at 80 min was over 60%.

### Example 60: Dissolution of CAP-DMG-Na-Lopinavir (32%)-Ritonavir (8%) ASD

Drug release percentages of CAP-DMG-Na-Lopinavir (32%)-Ritonavir (8%) ASD at 80 min was over 80%.

### Example 61: Non-Sink Powder Dissolution

Non-sink powder dissolution was conducted in pH 6.8, 50 mM phosphate sodium buffer in the beaker with paddle rotating at 100 rpm per minute. An in-situ Rainbow fiber optic ultraviolet spectrometer with a fiber optics (Pion, Billerica, MA, USA) was used to monitor drug concentration over time. 10 mm and 5 mm fiber optics were for Ledipasvir and Felodipine respectively. Second derivative analysis was applied to correct the spectral baseline and a calibration curve of area under curve (AUC) of different range of wavelength were used to calculate the drug concentration. 410-420 nm and 320-340 nm were for Ledipasvir and Felodipine respectively.

### Example 62: Dissolution of PVAP-DMG-Na-Felodipine ASDs

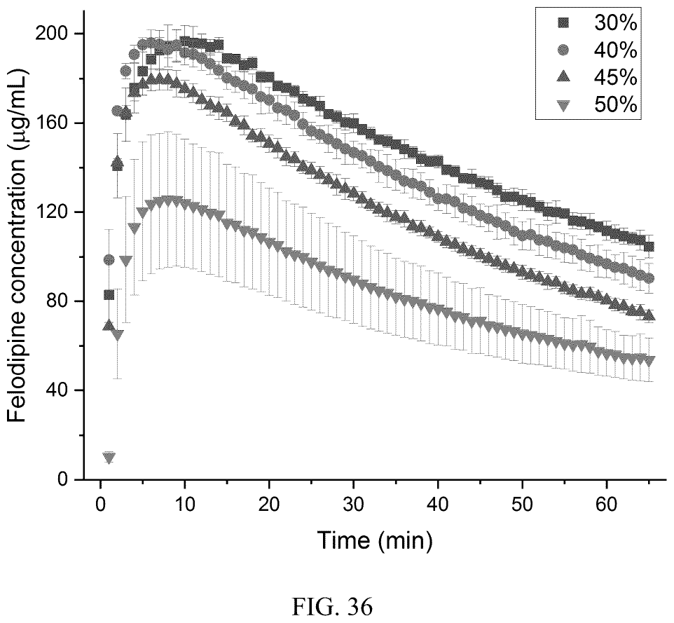

Felodipine release profile of PVAP-DMG-Na-Felodipine ASDs at different drug loadings is shown in FIG. 36. Maximum concentration if all drug releases from the ASD is 200 μg/mL in all cases.

### Example 63: Dissolution of PVAP-DMG-Na-Ledipasvir ASDs

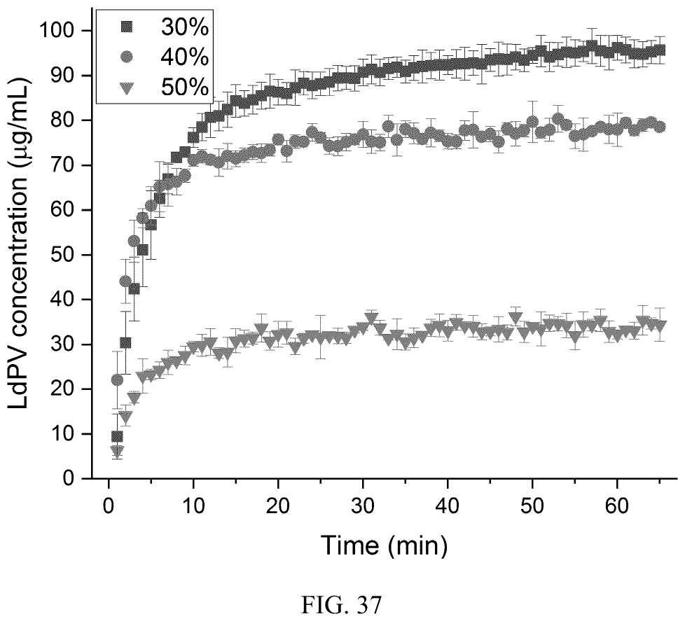

Ledipasvir release profile of PVAP-DMG-Na-Ledipasvir ASDs at different drug loadings is shown in FIG. 37. Maximum concentration if all drug releases from the ASD is 100 μg/mL in all cases.

### Example 64: Characterization of ASDs

### Glass Transition Temperature (T_{g}) by DSC and DMA

T_{g} values of neat polymers and ASDs were measured by either DSC or DMA with results summarized in Table 8 below.

TABLE 8

| Neat | | | | | |
|---|---|---|---|---|---|
| polymer | PAMP | PTHAM | PMP | PBTP | PDIP |
| DSC | 68.2 (1.9) | 54.9 (1.7) | 58.1 (1.4) | 84.5 (0.9) | 78.1 (1.8) |
| onset | | | | | |
| T_{g} (° C.) | | | | | |
| Neat | | | | | |
| polymer | PBTM | PTEA | PTBA | HP-50 | HP-Na |
| DSC | — | 84.0 (1.7) | 92.1 (2.1) | 127.1 (2.7) | — |
| onset | | | | | |
| T_{g} (° C.) | | | | | |
| DMA | 135.7 (2.1) | — | — | 172.8 (0.4) | 231.3 (2.8) |
| peak T_{g} | | | | | |
| (° C.)^{a} | | | | | |
| ASD | | | | | |
| polymer | HP-50 | HP-Na | PTEA | HP-Na | HP-Na |
| (% DL) | (20%) | (20%) | (20%) | (30%) | (50%) |
| DSC | 83.4 (0.4) | — | 77.2 (0.9) | — | — |
| onset | | | | | |
| T_{g} (° C.) | | | | | |
| DMA | 142.0 (2.0) | 216.2 (0.6) | 116.7 (1.1) | 207.9 (1.0) | 200.8 (1.0) |
| peak | | | | | |
| T_{g} (° C.) | | | | | |

^{a}Peak of tan δ was used.
Standard errors of the mean are shown in parentheses, where n = 3.

### Glass Transition Temperature (T_{g}) by DSC

TABLE 9
Summary of glass transition temperature of neat polymers and Miconazole ASDs

| | | | Miconazole | onset | Standard |
|---|---|---|---|---|---|
| ASD | | Amount | Loading | T_{g} | error |
| polymer | Cation precursor | (equiv)^{a} | (% w/w) | (° C.) | (n = 3) |
| HP-50 | Ammediol | 1 | 0 | 63.2 | 2.1 |
| HP-50 | Meglumine | 1 | 0 | 53.6 | 1.0 |
| HP-50 | DIPA | 1 | 0 | 110.2 | 1.3 |
| | (Diisopropylamine) | | | | |
| HP-50 | BIS-TRIS | 1 | 20 | 39.7 | 1.5 |
| HP-50 | Tris base | 1 | 20 | 53.7 | 0.2 |
| HP-50 | Tris base | 0.8 | 20 | 58.5 | 0.5 |
| HP-50 | Tris base | 1 | 30 | 43.1 | 0.5 |
| HP-50 | Tris base | 1 | 35 | 38.7 | 1.1 |
| HP-50 | Tris base | 1 | 40 | 34.1 | 0.6 |
| HP-50 | Tris base | 1 | 60 | 19.8 | 0.1 |
| HP-50 | DIPEA | 1 | 20 | 73.7 | 0.9 |
| HP-50 | AMP | 1 | 20 | 25.1 | 1.3 |
| HP-50 | — | — | 20 | 83.4 | 0.4 |
| HP-50 | — | — | 30 | 69.7 | 1.8 |
| HP-50 | — | — | 40 | 55.4 | 0.2 |
| HP-50 | Triethanolamine | 1 | 20 | 13.6 | 0.6 |
| HP-50 | DIPA | 1 | 20 | 64.0 | 0.4 |
| HP-50 | DIPA | 1 | 30 | 49.2 | 1.1 |
| HP-50 | DIPA | 1 | 40 | 37.9 | 0.3 |
| HP-50 | DIPA | 1 | 60 | 17.5 | 0.9 |
| HP-50 | TEA | 1 | 20 | 77.2 | 0.9 |
| HP-50 | TEA | 1 | 30 | 58.1 | 1.0 |
| HP-50 | Ammediol | 1 | 20 | 37.1 | 0.9 |
| HP-50 | Ammediol | 1 | 30 | 28.6 | 2.4 |
| HP-50 | Ammediol | 1 | 40 | 23.9 | 0.6 |
| HP-50 | Ammediol | 1 | 60 | 13.0 | 2.0 |
| HP-50 | Ammediol | 1 | 65 | 10.2 | 1.5 |
| HP-50 | Meglumine | 1 | 60 | 17.3 | 1.2 |
| HP-50 | Meglumine | 1 | 70 | 7.8 | 0.1 |
| HP-50 | Meglumine | 0.3 | 20 | 73.6 | 0.4 |
| HP-50 | Meglumine | 0.7 | 20 | 65.1 | 0.2 |
| HP-50 | Meglumine | 0.45 | 20 | 71.3 | 1.0 |
| HP-50 | MDEA | 1 | 20 | 25.8 | 2.6 |
| HP-50 | MDEA | 1 | 30 | 15.8 | 2.1 |
| HP-50 | MDEA | 1 | 60 | 8.7 | 1.6 |
| HP-50 | DEAE | 1 | 20 | 31.2 | 2.1 |
| HP-50 | DEAE | 1 | 50 | 8.0 | 1.6 |
| HP-50 | DMEA | 1 | 50 | 18.1 | 1.2 |
| HP-50 | DMEA | 1 | 60 | 11.6 | 1.4 |
| HP-50 | Proline-NH₄ | 1 | 65 | 17.7 | 1.4 |
| HP-55 | Ammediol | 1 | 40 | 27.0 | 1.2 |
| Eudragit | Ammediol | 1 | 40 | 115.5 | 0.4 |
| L100 | | | | | |
| CAP | Ammediol | 1 | 40 | 29.1 | 0.8 |
| AS-MF | Ammediol | 1 | 40 | 24.5 | 0.9 |
| AS-LF | Ammediol | 1 | 40 | 24.0 | 0.3 |

^{a}Amount of cation precursor to carboxylic acid in acidic polymer used in the ASD.

1. A composition comprising a polymer salt that comprises a main chain and one or more negatively charged carboxylic groups in the main chain, and one or more counterions of said carboxylates.

2. The composition of claim 1, wherein the polymer is at least one polymer selected from the group consisting of: methacrylic acid/ethyl acrylate copolymer; methacrylic acid/methyl methacrylate copolymer; methacrylic acid copolymer; hydroxypropyl methylcellulose acetate succinate (HPMCAS); hydroxypropyl methyl phthalate (HPMCP); cellulose acetate phthalate (CAP); cellulose acetate trimellitate; cellulose acetate succinate; methyl cellulose phthalic acid; hydroxymethyl cellulose ethyl phthalate; hydroxypropyl methyl acetic acid; maleic acid ester; hydroxypropyl methyl trimellitate; carboxy methyl ethyl cellulose; polyvinyl butyrate; polyvinyl alcohol acetate phthalate; polyvinyl acetate phthalate (PVAP), poly(acrylic acid) (PAA) and a combination thereof.

3. The composition of claim 1, wherein the one or more counterions are at least one salt cation selected from the group consisting of: a Group 1 metal cation; ammonium comprises a formula R¹R²R³R⁴N⁺, wherein each of R¹, R², R³ and R⁴ is independently a hydrogen or alkyl group or an aryl group; and a combination thereof.

4. The composition of claim 3, wherein the Group 1 metal cation is at least one selected from the group consisting of lithium cation (Li⁺), sodium cation (Na⁺), potassium cation (K⁺), rubidium cation (Rb⁺), caesium cation (Cs⁺), and a combination thereof.

5. The composition of claim 3, wherein the ammonium is selected from NH₄⁺; R⁴ is hydrogen and R¹R²R³N is from meglumine, tris base, triethanolamine, 2-dimethylaminoethanol, triethylamine, ammediol, glucosamine; R¹R²R³R⁴N+ is selected from choline.

6. An amorphous solid dispersion (ASD) composition comprising:
an active pharmaceutical agent (API); and
a polymer salt of any of claims 1-5.

7. The ASD composition of claim 6, further comprising an amino acid or an amino sulfonic acid.

8. The ASD composition of claim 6, wherein the polymer salt comprises up to about 90 wt % of the ASD composition.

9. The ASD composition of claim 6, wherein the API comprises at least about 5 wt % of the ASD composition.

10. The ASD composition of claim 6, wherein the API is at least one selected from the group consisting of: antihypertensive agents, antianxiety agents, anticlotting agents, anticonvulsant agents, blood glucose-lowering agents, decongestant agents, antihistamine agents, antitussive agents, antineoplastic agents, beta blocker agents, anti-inflammatory agents, antipsychotic agents, cognitive enhancer agents, anti-atherosclerotic agents, cholesterol-reducing agents, anti-obesity agents, autoimmune disorder agents, anti-impotence agents, antibacterial agents, antifungal agents, hypnotic agents, anti-Parkinsonism agents, anti-Alzheimer's disease agents, antibiotic agents, anti-depressant agents, antiviral agents, glycogen phosphorylase inhibitor agents, cholesterol ester transfer protein inhibitor agents, and a combination thereof.

11. The ASD composition of claim 9, wherein the API is at least one selected from the group consisting of: miconazole, lopinavir, ledipasvir, ritonavir, clotrimazole, felodipine, and a combination thereof.

12. The ASD composition of claim 8, wherein the API comprises 10 wt % or more of the ASD composition and the polymer salt comprises up to about 90 wt % of the ASD composition.

13. A method for making a polymer salt, the method comprising:
providing a polymer,
adding a base to react with one or more carboxylic acid groups in a main chain of the polymer to thereby convert the polymer into a polymer salt, wherein the base is used in an amount of 0.1 equiv to 1.0 equiv, in relation to one equivalent of carboxylic acid groups in the polymer.

14. The method of claim 13, wherein a salt of a zwitterion is used as the base, which is selected from a salt of an amino acid or an amino sulfonic acid.

15. The method of claim 14, wherein the method is performed without an isolation step.

16. A method for making a polymer salt ASD, the method comprising:
making a polymer salt in the method of any of claim 13; and
combining the produced polymer salt with an API to form an ASD composition.

17. The method of claim 16, wherein the polymer for salt preparation is at least one polymer is selected from the group consisting of: methacrylic acid/ethyl acrylate copolymer; methacrylic acid/methyl methacrylate copolymer; methacrylic acid copolymer; hydroxypropyl methylcellulose acetate succinate (HPMCAS); hydroxypropyl methyl phthalate (HPMCP); cellulose acetate phthalate (CAP); cellulose acetate trimellitate; cellulose acetate succinate; methyl cellulose phthalic acid; hydroxymethyl cellulose ethyl phthalate; hydroxypropyl methyl acetic acid; maleic acid ester; hydroxypropyl methyl trimellitate; carboxy methyl ethyl cellulose; polyvinyl butyrate; polyvinyl alcohol acetate phthalate; polyvinyl acetate phthalate (PVAP), poly(acrylic acid) (PAA) and a combination thereof.

18. The method of claim 16, wherein a salt cation of the polymer salt is at least one salt cation selected from the group consisting of: a Group 1 metal cation; ammonium comprises a formula R¹R²R³R⁴N⁺, wherein each of R¹, R², R³ and R⁴ is independently a hydrogen or alkyl group or an aryl group; and a combination thereof.

19. The method of claim 16, wherein the Group 1 metal cation is at least one selected from the group consisting of lithium cation (Li⁺), sodium cation (Na⁺), potassium cation (K⁺), rubidium cation (Rb⁺), caesium cation (Cs⁺), and a combination thereof.

20. The method of claim 16, wherein the API is at least one selected from the group consisting of: antihypertensive agents, antianxiety agents, anticlotting agents, anticonvulsant agents, blood glucose-lowering agents, decongestant agents, antihistamine agents, antitussive agents, antineoplastic agents, beta blocker agents, anti-inflammatory agents, antipsychotic agents, cognitive enhancer agents, anti-atherosclerotic agents, cholesterol-reducing agents, anti-obesity agents, autoimmune disorder agents, anti-impotence agents, antibacterial agents, antifungal agents, hypnotic agents, anti-Parkinsonism agents, anti-Alzheimer's disease agents, antibiotic agents, anti-depressant agents, antiviral agents, glycogen phosphorylase inhibitor agents, cholesterol ester transfer protein inhibitor agents, and a combination thereof.

21-31. (canceled)
